(19) United States
(12) Patent Application Publication
(10) Pub. No.: US 2023/0157548 A1
(43) Pub. Date: May 25, 2023

(54) SYSTEM AND METHOD FOR DETECTION OF RESIDUAL CANCEROUS TISSUE

(71) Applicant: Nutek O.I.D.O Ltd., Rishon-LeZion (IL)

(72) Inventors: Ofer BRAUN, Zoran (IL); Dov COHEN, Rishon Le Zion (IL); Ilan Avital ZORMAN, Ramat HaSharon (IL)

(73) Assignee: Nutek O.I.D.O Ltd., Rishon-LeZion (IL)

(21) Appl. No.: 17/912,507

(22) PCT Filed: Mar. 18, 2021
(86) PCT No.: PCT/IL2021/050301

§ 371 Date: Sep. 18, 2022

Related U.S. Application Data
(60) Provisional application No. 63/067,896, filed on Aug. 20, 2020.
(60) Provisional application No. 62/991,100, filed on Mar. 18, 2020.

(51) Int. Cl. A61B 5/00

(52) U.S. Cl. CPC A61B 5/0075; A61B 2560/0431; A61B 2576/00

(57) ABSTRACT

A probe for detecting cancerous tissue, the probe including an illuminator, an optical spectrum analyzer, and an indicator for indicating whether cancerous tissue has been detected in reflected spectrum analyzed by the optical spectrum analyzer. A method for detecting cancerous tissue, the method including providing a probe including an optical spectrum analyzer, placing the probe to include tissue-to-be-tested within a Field-Of-View (FOV) of the spectrum analyzer, optically illuminating the tissue-to-be-tested, analyzing light reflected from the tissue-to-be-tested, and determining whether or not cancerous tissue is detected in the FOV. Related apparatus and methods are also described.

### RELATED APPLICATION/S

This application is a PCT Patent application claiming priority from U.S. Provisional Patent Application No. 63/067,896 filed on 20 Aug. 2020, and from U.S. Provisional Patent Application No. 62/991,100 filed on 18 Mar. 2020.

The contents of all of the above applications are incorporated by reference as if fully set forth herein.

### FIELD AND BACKGROUND

The present disclosure, in some embodiments thereof, relates to systems and methods for detection of residual cancerous tissue, and, more particularly, but not exclusively, to systems and methods for optical detection of residual cancerous tissue, and more particularly, but not exclusively, to systems and methods for optical detection of residual cancerous tissue and marking location of the residual cancerous tissue.

Optical spectroscopy is a technique that is exquisitely sensitive to detect malignant tissue. There have been many reports about the use of optical spectroscopy for detecting cancerous tissue, but only a few have employed spectral analysis algorithms that extract quantitative, physically meaningful parameters from the tissue spectra. Quantitative optical spectroscopy in the UV-visible wavelength regime has been used in the study of cancers, including the breast and cervix, by variety researchers. While challenges in implementation remain, these methods may provide value in characterizing important biomarkers of cancer during various clinical setting such as during operation.

Additional background art includes:

An article by Brown J. Q., Vishwanath K., Palmer G. M. and Ramanujam N. published in 2009, titled “Advances in quantitative UV-visible spectroscopy for clinical and pre-clinical application in cancer”, published in Current Opinion in Biotechnology, 20:119-131;

An article titled “Review of methods for intraoperative margin detection for breast conserving surgery”, published in Journal of Biomedical Optics 23(10), 100901 (October 2018);

An article titled “Diagnosis of breast cancer using elastic-scattering spectroscopy: preliminary clinical results”, published in Journal of Biomedical Optics 5(2), 221-228 (April 2000);

An article titled “Detecting positive surgical margins: utilization of light-reflectance spectroscopy on ex vivo prostate specimens” published in BJU Int 2016; 118: 885-889;

A publication titled “Hyperspectral prism-grating-prism imaging spectrograph”, written by Mauri Aikio of VTT (Technical Research Centre of Finland) in a dissertation for the degree of Doctor of Technology at the University of Oulu; and

An article by M. A. Aswathy, M. Jagannath, titled “Detection of breast cancer on digital histopathology images: Present status and future possibilities”, published in Informatics in Medicine Unlocked 8 (2017) 74-79.

The disclosures of all references mentioned above and throughout the present specification, as well as the disclosures of all references mentioned in those references, are hereby incorporated herein by reference.

### SUMMARY

The present disclosure, in some embodiments thereof, describes systems and methods for detection of residual cancerous tissue, and for guiding a physician where the residual cancerous tissue is located.

The following embodiments and aspects thereof are described and illustrated in conjunction with systems, tools and methods which are meant to be exemplary and illustrative, not limiting in scope.

There is provided, in accordance with an embodiment, a probe configured for real-time detection of residual cancerous tissue having a casing configured to be gripped in a hand of a user, an optical system configured to obtain data from energy of reflected light reflecting from tissue at which the optical system is directed, an indication unit configured to provide an indication whether a scanned area of an operation cavity includes residual cancerous tissue, at least one processor configured receive data from the optical system, execute a spectral analysis of the data, determine a scanned area includes residual cancerous tissue, designate a location in scanned area in which the residual cancerous tissue has been detected according to the data, and generate an indication that an area includes residual cancerous tissue, and having an indicator configured to notify the user of the indication generated by the at least one processor.

In certain embodiments, the probe further includes a stamping tool configured to mark the location in which the residual cancerous tissue was detected.

In certain embodiments, the probe further includes a display unit configured to display a two-dimensional analysis superimposed over an image of the scanned area to facilitate guiding the user to a location of the residual cancerous tissue.

In certain embodiments, the optical system includes an emitter configured to emit a light in a direction of the tissue, a sensor unit configured to detect the energy of the reflected light reflected from the tissue, and an assortment of optical devices configured to guide the light from the emitter to the tissue and the reflected light from the tissue to the sensor.

There is also provided, in accordance with an embodiment, a scanning system incorporating spectral analysis, to identify and designate the location of residual cancerous tissue a) within an operational cavity on patient's body, b) on the margins of a resected tumor mass.

There is also provided, in accordance with an embodiment, an intraoperative method to guide the surgeon to the location of residual cancerous tissue a) within an operational cavity on patient's body, after removing the cancerous tumor mass, b) on the margins of a resected tumor mass.

There is also provided, in accordance with an embodiment, a method of marking the perimeters of cancerous tissue a) within an operational cavity on the patient's body, b) on the margins of a resected tumor mass.

There is also provided, in accordance with an embodiment, a method to process the spectral data in real time.

There is also provided, in accordance with an embodiment, a method of presenting 2D spectral analysis results of a scanned area superimposed over 2D spatial image of the same area. May optionally be stored in the sensor memory available for download after the operation for record or debriefing.

There is provided, in accordance with an embodiment, a probe that assists the surgeon in completely remove residual cancerous tissues on patient's body after removing the tumor mass, supports following objectives: eliminating the major cause for reoperation, reduction in patient's recovery time, reduction in recurrence probability, eliminating the impact of variability among surgeons by providing an objective tool.

In addition to the exemplary aspects and embodiments described above, further aspects and embodiments will become apparent by reference to the figures and by study of the following detailed description.

According to an aspect of some embodiments of the present disclosure there is provided a probe for real-time detection of residual cancerous tissue, including a casing configured to be gripped in a hand of a user, an optical system configured to obtain data from energy of reflected light reflecting from tissue at which the optical system is directed, an indication unit configured to provide an indication whether a scanned area of an operation cavity includes residual cancerous tissue, at least one processor configured to receive data from the optical system, execute a spectral analysis of the data, determine a scanned area includes residual cancerous tissue, designate a location in scanned area in which the residual cancerous tissue has been detected according to the data, generate an indication that an area includes residual cancerous tissue, an indicator configured to notify the user of the indication generated by the at least one processor.

According to some embodiments of the disclosure, further including a stamping tool configured to mark the location in which the residual cancerous tissue was detected.

According to some embodiments of the disclosure, further including a display unit configured to display a two dimensional analysis superimposed over an image of the scanned area to facilitate guiding the user to a location of the residual cancerous tissue.

According to some embodiments of the disclosure, the optical system includes an emitter configured to emit a light in a direction of the tissue, a sensor unit configured to detect the energy of the reflected light reflected from the tissue, and an assortment of optical devices configured to guide the light from the emitter to the tissue and the reflected light from the tissue to the sensor.

According to an aspect of some embodiments of the present disclosure there is provided a scanning system incorporating spectral analysis, to identify and designate the location of residual cancerous tissue a) within an operational cavity on patient's body, b) on the margins of a resected tumor mass.

According to an aspect of some embodiments of the present disclosure there is provided an intraoperative method to guide the surgeon to the location of residual cancerous tissue a) within an operational cavity on patient's body, after removing the cancerous tumor mass, b) on the margins of a resected tumor mass.

According to an aspect of some embodiments of the present disclosure there is provided a method of marking the location of cancerous tissue a) within an operational cavity on the patient's body, b) on the margins of a resected tumor mass.

According to an aspect of some embodiments of the present disclosure there is provided a method to process the spectral data in real time.

According to an aspect of some embodiments of the present disclosure there is provided a method of presenting two dimensional spectral analysis results of a scanned area superimposed over two dimensional spatial image of the same area. May optionally be stored in the sensor memory available for download after the operation for record or debriefing.

According to an aspect of some embodiments of the present disclosure there is provided a probe for real-time detection of residual cancerous tissue, including a casing configured to be gripped in a hand of a user, an optical system configured to obtain data from energy of reflected light reflecting from tissue at which the optical system is directed, an indication unit configured to provide an indication whether a scanned area of an operation cavity includes residual cancerous tissue, at least one processor configured receive data from the optical system, execute a spectral analysis of the data, determine a scanned area includes residual cancerous tissue, designate a location in scanned area in which the residual cancerous tissue has been detected according to the data, generate an indication that an area includes residual cancerous tissue, and an indicator configured to notify the user of the indication generated by the at least one processor.

According to some embodiments of the disclosure, further including a stamping tool configured to mark the location in which the residual cancerous tissue was detected.

According to some embodiments of the disclosure, further including a display unit configured to display a two dimensional analysis superimposed over an image of the scanned area to facilitate guiding the user to a location of the residual cancerous tissue.

According to some embodiments of the disclosure, the optical system includes an emitter configured to emit a light in a direction of the tissue, a sensor unit configured to detect the energy of the reflected light reflected from the tissue, and an assortment of optical devices configured to guide the light from the emitter to the tissue and the reflected light from the tissue to the sensor.

According to some embodiments of the disclosure, further including a scanning system configured to facilitate spectral analysis to identify and designate the location of residual cancerous tissue within an operational cavity in a body of a patient and on margins of a resected tumor mass.

According to an aspect of some embodiments of the present disclosure there is provided a method including using at least one hardware processor for guiding a surgeon to a location of residual cancerous tissue within an operational cavity in a body of a patient after removing a cancerous tumor mass and on margins of a resected tumor mass.

According to an aspect of some embodiments of the present disclosure there is provided a method including using at least one hardware processor for marking the perimeters of cancerous tissue an operational cavity in a body of a patient after removing a cancerous tumor mass and on margins of a resected tumor mass.

According to an aspect of some embodiments of the present disclosure there is provided a method including using at least one hardware processor for analyzing spectral data in real time.

According to an aspect of some embodiments of the present disclosure there is provided a method including using at least one hardware processor for presenting two-dimensional spectral analysis results of a scanned area superimposed over two-dimensional spatial image of the same area, and storing in the two-dimensional spectral analysis in a sensor memory.

According to an aspect of some embodiments of the present disclosure there is provided a method including using at least one hardware processor for obtaining an image of a scanned area at a predetermined pixel resolution, performing a spectral analysis at a pixel level of the image, obtaining a spectral signature for each pixel of the image, determining a morphological differentiation to differentiate between cancerous tissue and benign tissue, generating an indication of the cancerous tissue according to the spectral signature and the morphological differentiation.

According to some embodiments of the disclosure, the analysis is performed by artificial intelligence and machine learning algorithms.

According to some embodiments of the disclosure, further including storing structural images of benign and cancerous tissue in a database, and, wherein the analysis of the image is achieved through reference to the structural images.

According to some embodiments of the disclosure, wherein a pixel is designated according to an (X, Y) coordinate associated with the image.

According to an aspect of some embodiments of the present disclosure there is provided a probe for detecting cancerous tissue, the probe including an illuminator, an optical spectrum analyzer, and an indicator for indicating whether cancerous tissue has been detected in reflected spectrum analyzed by the optical spectrum analyzer.

According to some embodiments of the disclosure, the optical spectrum analyzer includes a hyperspectral analyzer.

According to some embodiments of the disclosure, the probe includes a detachable probe head detachable from a handle of the probe, and wherein the probe head includes a component for contacting tissue.

According to some embodiments of the disclosure, the probe includes a disposable probe head detachable from a handle of the probe, and wherein the probe head includes a component for contacting tissue.

According to some embodiments of the disclosure, the probe includes a window for transferring reflected light from illuminated tissue to the spectrum analyzer.

According to some embodiments of the disclosure, the probe includes a stamp for marking tissue at a border of the window. According to some embodiments of the disclosure, the probe includes a stamp for marking tissue at a plurality of borders of the window.

According to some embodiments of the disclosure, the probe includes an actuator for automatically stamping tissue based on whether cancerous tissue has been detected in reflected spectrum analyzed by the optical spectrum analyzer.

According to some embodiments of the disclosure, the stamp is configured to stamp tissue based on where in the FOV cancerous tissue has been detected.

According to some embodiments of the disclosure, the indicator includes indicator lights for indicating one or more of scan in progress, cancerous tissue detected, and no cancerous tissue detected.

According to some embodiments of the disclosure, configured to display images captured by the optical spectrum analyzer.

According to some embodiments of the disclosure, the detachable probe head includes a window for transferring reflected light from illuminated tissue to the spectrum analyzer.

According to some embodiments of the disclosure, the probe is configured to detach the detachable probe head at the window.

According to some embodiments of the disclosure, the probe is configured to detach the detachable probe head at a slit along the optical path from the window to the spectrum analyzer.

According to some embodiments of the disclosure, the probe is configured to detach the detachable probe head at location along the optical path from the window to the spectrum analyzer where optical rays are parallel.

According to some embodiments of the disclosure, the probe includes a battery for handheld operation.

According to an aspect of some embodiments of the present disclosure there is provided a kit including a probe and a probe base, where the probe and the probe base are configured for the probe base to charge the probe battery.

According to some embodiments of the disclosure, further including a display for displaying images captured by the probe.

According to an aspect of some embodiments of the present disclosure there is provided a method for detecting cancerous tissue, the method including providing a probe including an optical spectrum analyzer, placing the probe to include tissue-to-be-tested within a Field-Of-View (FOV) of the spectrum analyzer, optically illuminating the tissue-to-be-tested, and analyzing light reflected from the tissue-to-be-tested, and determining whether or not cancerous tissue is detected in the FOV.

According to some embodiments of the disclosure, the analyzing includes diffuse spectroscopy analysis.

According to some embodiments of the disclosure, the determining includes analyzing a spectrum of each pixel in the FOV, determining a type of tissue in the pixel, allocating a value to the pixel based on the type of tissue, producing a synthetic image from the values of the pixels, and performing image analysis of the synthetic image.

According to some embodiments of the disclosure, the determining includes image analysis of images captured at a specific wavelength band.

According to some embodiments of the disclosure, the determining includes image analysis of a plurality of images of the FOV captured at a plurality of wavelength bands.

According to some embodiments of the disclosure, the plurality of wavelength bands is a group selected from an entire range wavelengths bands reflected from the tissue-to-be-tested.

According to some embodiments of the disclosure, machine learning is used to select the plurality of wavelength bands.

According to some embodiments of the disclosure, the determining includes combining a likelihood of detection of cancerous tissue based on image analysis and a likelihood of detection of cancerous tissue based on diffuse spectroscopy analysis.

According to some embodiments of the disclosure, the analysis is performed on an imaged area of a size of a single tissue cell.

According to some embodiments of the disclosure, placing the probe includes placing the probe to flatten the tissue-to-be-tested.

According to some embodiments of the disclosure, further including performing autofocusing of the light reflected from the tissue-to-be-tested.

According to some embodiments of the disclosure, placing the probe includes placing a window included in the probe against the tissue to flatten the tissue-to-be-tested.

According to some embodiments of the disclosure, further including marking an area of the FOV on the tissue.

According to some embodiments of the disclosure, the marking includes automatically marking.

According to some embodiments of the disclosure, the marking includes automatically marking differently if cancerous tissue has been detected in the FOV than if cancerous tissue has not been detected in the FOV.

According to some embodiments of the disclosure, the marking indicates where within the FOV cancerous tissue has been detected.

According to some embodiments of the disclosure, further including indicating one or more of scan in progress, cancerous tissue detected, and no cancerous tissue detected.

According to an aspect of some embodiments of the present disclosure there is provided a method for detecting cancerous tissue, the method including providing a probe including an optical spectrum analyzer, placing the probe to include tissue-to-be-tested within a Field-Of-View (FOV) of the spectrum analyzer, optically illuminating the tissue-to-be-tested, and using diffuse spectroscopy analysis to analyze light reflected from an area of a single tissue cell, thereby determining whether or not cancerous tissue is detected in the single tissue cell.

According to an aspect of some embodiments of the present disclosure there is provided a method for detecting cancerous tissue, the method including providing a probe including an imaging sensor, placing a window of the probe to flatten tissue-to-be-tested within a Field-Of-View (FOV) of the spectrum analyzer, optically illuminating the tissue-to-be-tested, and using image analysis to analyze images captured by the imaging sensor from an area of a single tissue cell, thereby determining whether or not cancerous tissue is detected in the single tissue cell.

According to an aspect of some embodiments of the present disclosure there is provided a method for marking cancerous tissue, the method including providing a probe including an optical spectrum analyzer, placing the probe to include tissue-to-be-tested within a Field-Of-View (FOV) of the spectrum analyzer, optically illuminating the tissue-to-be-tested, analyzing light reflected from the tissue-to-be-tested, determining whether or not cancerous tissue is detected in the FOV, and marking an area within the FOV on the tissue where cancerous tissue has been detected.

According to an aspect of some embodiments of the present disclosure there is provided a method for guiding scan of tissue, the method including providing a probe including an optical spectrum analyzer, placing the probe to include tissue-to-be-tested within a Field-Of-View (FOV) of the spectrum analyzer, and marking an area of the FOV on the tissue.

According to an aspect of some embodiments of the present disclosure there is provided a method for guiding scan of tissue, the method including providing a probe including an optical spectrum analyzer, marking an area of tissue-to-be-tested using a color visible to the spectrum analyzer, placing the probe to include tissue-to-be-tested within a Field-Of-View (FOV) of the spectrum analyzer, and displaying images captured by the probe.

According to some embodiments of the disclosure, the displaying includes displaying the marking.

According to some embodiments of the disclosure, further including indicating whether all of the area of tissue-to-be-tested has been imaged by the probe.

According to some embodiments of the disclosure, the displaying includes displaying where cancerous tissue has been detected.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present disclosure may be embodied as a system, method or computer program product. Accordingly, some embodiments of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present disclosure may take the form of a computer program product embodied in one or more computer readable medium(s) having computer readable program code embodied thereon. Implementation of the method and/or system of some embodiments of the disclosure can involve performing and/or completing selected tasks manually, automatically, or a combination thereof. Moreover, according to actual instrumentation and equipment of some embodiments of the method and/or system of the disclosure, several selected tasks could be implemented by hardware, by software or by firmware and/or by a combination thereof, e.g., using an operating system.

For example, hardware for performing selected tasks according to some embodiments of the disclosure could be implemented as a chip or a circuit. As software, selected tasks according to some embodiments of the disclosure could be implemented as a plurality of software instructions being executed by a computer using any suitable operating system. In an exemplary embodiment of the disclosure, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as detection of residual cancerous tissue, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

Exemplary embodiments are illustrated in referenced figures. Dimensions of components and features shown in the figures are generally chosen for convenience and clarity of presentation and are not necessarily shown to scale. The figures are listed below.

In the drawings:

FIG. 1A is a simplified schematic illustration of a probe and a tissue to be scanned according to an example embodiment;

FIG. 1B is a simplified schematic illustration of a probe and a hosting cradle, according to according to an example embodiment;

FIG. 1C is a simplified schematic illustration of a probe and an external display, according to according to an example embodiment;

FIG. 2 is a simplified schematic illustration of elements and indicators of a probe according to some example embodiments;

FIG. 3A is a simplified schematic illustration of a scanning window with surrounding stamping elements, according to some example embodiments;

FIG. 3B is a simplified schematic illustration of a scanning window with surrounding stamping elements, according to some example embodiments;

FIG. 3C is a simplified schematic illustration of a scanning window with surrounding stamping elements, according to some example embodiments;

FIG. 3D is a simplified schematic illustration of a display showing areas which have been scanned, according to some example embodiments;

FIG. 4A is a simplified schematic illustration of a method of operating a probe according to an example embodiment;

FIG. 4B is a simplified schematic illustration of a method of operating a probe according to an example embodiment;

FIG. 4C is a simplified schematic illustration of a probe according to an example embodiment;

FIG. 5 is a simplified illustration of a probe scanning tissue and data produced by the probe, according to an example embodiment;

FIG. 6A is a simplified illustration of an optical design of a probe according to an example embodiment;

FIGS. 6B and 6C are simplified illustrations of methods for marking cancerous tissue according to some example embodiments;

FIGS. 7A-7C show a method of processing sensor data according to example embodiments;

FIG. 8 is a simplified illustration of a spectral graph and different wavelength spatial images which were used to produce the graph according to an example embodiment;

FIG. 9 is a graph showing experimental spectra of several locations in tissue scanned according to an example embodiment;

FIG. 10 is a simplified flow chart illustration of a method for detecting cancerous tissue according to an example embodiment;

FIG. 11 is a simplified flow chart illustration of a method for marking cancerous tissue according to an example embodiment; and

FIG. 12 is a simplified flow chart illustration of a method for guiding scan of tissue according to an example embodiment.

### DESCRIPTION OF SPECIFIC EMBODIMENTS

The present disclosure, in some embodiments thereof, relates to systems and methods for detection of residual cancerous tissue, and, more particularly, but not exclusively, to systems and methods for optical detection of residual cancerous tissue, and more particularly, but not exclusively, to systems and methods for optical detection of residual cancerous tissue and marking location of the residual cancerous tissue.

Disclosed herein is a system and method for detecting residual cancerous tissue in real time, according to certain exemplary embodiments. The apparatus can be an intraoperative tool, based on spectral analysis. In some exemplary cases, the disclosed apparatus and method facilitate identifying residual cancerous tissue on a patient's body and guide a surgeon to the location of the residual cancerous tissues for complete removal. In other exemplary cases, the apparatus and method facilitate analyzing the resected tumor margins to assess whether the resected tumor margins are clean and free of cancerous tissue, which is indicative of a complete tumor removal, or whether the resected tumor margins are “not clean” and the tumor was not completely removed and therefore indicative that residual cancerous tissue may still exist within the operational cavity.

Overview

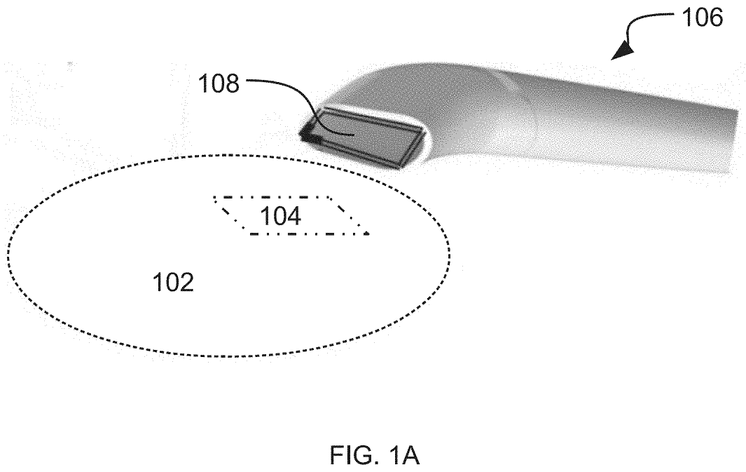

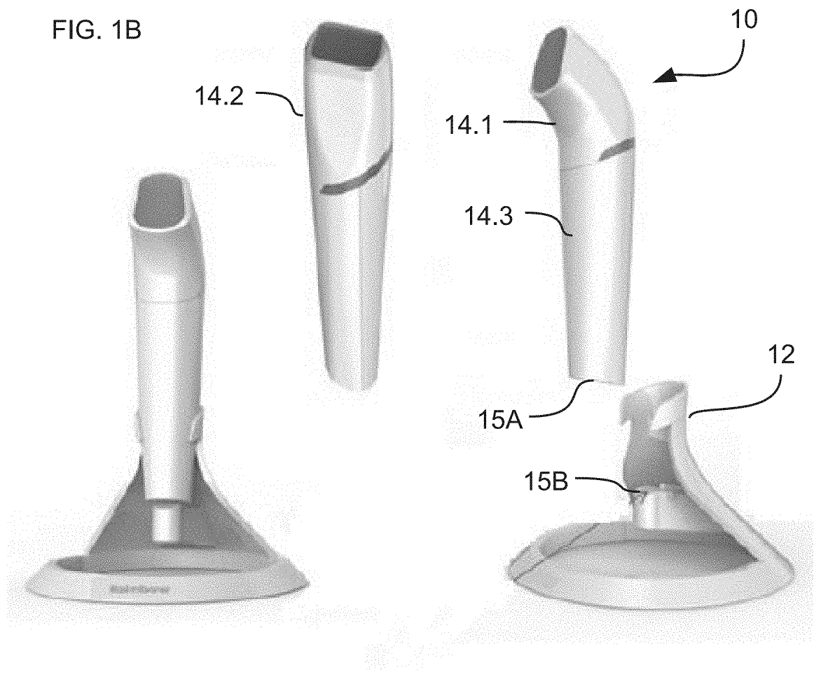

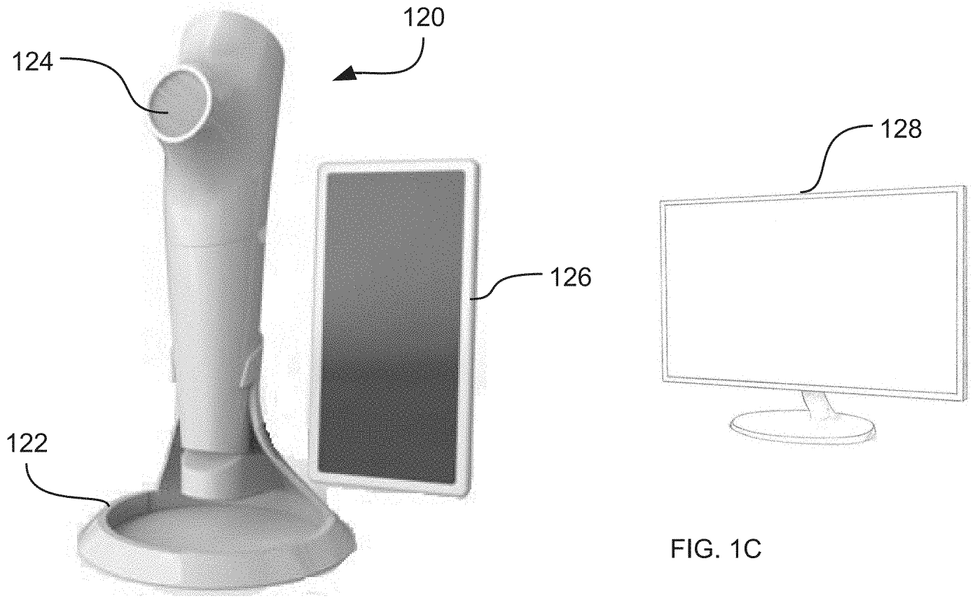

For purposes of better understanding some embodiments of the present disclosure, reference is first made to FIG. 1A, which is a simplified schematic illustration of a probe and a tissue to be scanned according to an example embodiment.

FIG. 1A shows an area of tissue 102 and a probe 106 for scanning the tissue 102. In a non-limiting example embodiment, a scanning window 108 in the probe 106 is placed upon the tissue 102. An area 104 of the tissue 102 is optically scanned, and reflected light is optionally analyzed to detect whether residual cancerous tissue remains in the area of tissue 102. After scanning the area 104, a physician moves the probe 106 to scan an additional area next to the area 104.

Taking the above as a general and non-limiting example, various aspects of the disclosure are introduced below.

Detection of Residual Cancerous Tissue

An aspect of some embodiments is related to detection of the residual cancerous tissue.

Spectral Analysis

In some embodiments, the detection is optionally performed by spectroscopic analysis of diffuse reflected light reflected from tissue.

In some embodiments, the reflected light is from one or more light source(s) built into a probe. In some embodiments, the light sources may be external to the probe. By way of a non-limiting example, the light source(s) may be a lamp used by a physician to illuminate the tissue upon which the physician is working. By way of a non-limiting example, the light source(s) may be a light source used by a physician during a laparoscopic procedure.

In some embodiments, the light source(s) may include a broad spectrum, or even white light. In some embodiments, the light source(s) may include wavelengths which excite fluorescence in biological matter.

In some embodiments, the detection is optionally performed by spectroscopic analysis in a range of 400-950 nanometers. Various wavelength ranges which are optionally implemented include 350-500 nanometers, 1,000-2,500 nanometers (in which light typically penetrates further into tissue) and 350-2500 nanometers.

In some embodiments, the spectroscopic analysis is performed on light measured in several wavelength ranges, or bands. The wavelength ranges may or may not be contiguous. By way of a non-limiting example, the spectroscopic analysis is performed at one, two, three, four, five, and so on up to 200 wavelengths bands.

In some embodiments, the wavelength bands are contiguous. When using contiguous wavelength bands, the spectroscopic analysis may fall into a category termed hyperspectral analysis.

In some embodiments, a range on one wavelength band may be 5 nanometers, or anywhere between 0.5 nanometers and 15, 20 and up to 50 nanometers.

In some embodiments, the spectroscopic analysis may aggregate analysis of several wavelength bands into one, using data from several wavelength bands as one.

In some embodiments, the detection is optionally performed by multi-spectral analysis of light reflected from tissue.

In some embodiments, the detection is optionally performed by hyperspectral analysis of light reflected from tissue. In some embodiments, the detection is optionally performed by hyperspectral analysis in a range of 400-950 nanometers. Various wavelength ranges which are optionally implemented include 350-500 nanometers, 1,000-2,500 nanometers (in which light typically penetrates further into tissue) and 350-2500 nanometers, divided into 110 wavelength bands, or two, three, four, five, and so on up to 200 wavelengths bands.

Hyperspectral analysis is typically performed at high spectral resolution, for example wavelength bandwidth of 5 nanometers, or in a range from 1 to 20 nanometers. Hyperspectral analysis typically uses contiguous wavelength bands.

In some embodiments, the spectroscopic analysis may aggregate analysis of several wavelength bands into one, using data from several wavelength bands as one.

In some embodiments, the detection is optionally based on a specific hyperspectral signature of cancerous cells.

In some embodiments, analyzing a spectrum of a spatial pixel identifies tissue type imaged by the pixel. In some embodiments, an aggregate of the spatial pixels may be used to produce a map or image of the identified tissue types. In some embodiments, the aggregate image is optionally displayed on an image display, built into a handheld probe and/or made available on a display separate from the probe.

In some embodiments, the identified tissue map is optionally displayed with “artificial” colors, also termed a “false color map” or a “pseudo color map”, with colors used to identify tissue type.

Image Analysis and/or Morphological Analysis

In some embodiments, the detection is optionally performed by image analysis and/or morphological analysis of diffuse reflected light reflected from tissue.

By way of a non-limiting example, such a method is described in above-mentioned publication titled “Detection of breast cancer on digital histopathology images: Present status and future possibilities”, published in Informatics in Medicine Unlocked 8 (2017) 74-79.

In some embodiments, the detection is optionally performed by image analysis and/or morphological analysis at a single spectral window, that is, by image analysis of an image captured at a single wavelength band, detecting cells which are detected as possibly cancerous.

In some embodiments, the detection is optionally performed by image analysis and/or morphological analysis on an image made up of more than one spectral window.

In some embodiments, the detection is optionally performed by image analysis at one or more wavelength(s) selected to provide detection, in order to reduce calculation and perform image analysis on less wavelength planes.

In some embodiments, a machine learning technique is optionally used to select which wavelength(s) are to be used for image analysis, in order to reduce calculation and perform image analysis on less wavelength planes.

In some embodiments, the detection is optionally performed by image analysis and/or morphological analysis on an image made up of a combination of all spectral windows.

In some embodiments, image analysis of a scanned area is performed, optionally at a pixel resolution. In some embodiments, a structure of the spatial image is optionally analyzed by an Artificial Intelligence and/or Machine Learning algorithm, to search for morphological structures indicating benign or cancerous tissue. In some embodiments, known structural images of benign and cancerous tissues are used as a data base for such analysis.

In some embodiments, the detection is optionally performed by spectral analysis of each spatial pixel. At each pixel, a type of tissue or a type of cell (fat, muscle, blood cell, brain cell) is optionally identified.

In some embodiments, a synthetic image is produced from the analyzed pixels. The synthetic image produced is an image of tissue types.

In some embodiments, the synthetic image is optionally displayed with “artificial” colors, also termed a “false color map” or a “pseudo color map”, with colors used to identify tissue type.

In some embodiments, the detection is optionally performed by image analysis of the morphology of the produced synthetic image.

Combining Spectral Analysis and Image Analysis and/or Morphological Analysis

In some embodiments, spectral analysis optionally identifies a spatial pixel which may belong to cancerous tissue, and image analysis and/or morphological analysis optionally analyzes an area of a spatial image surrounding the spatial pixel, potentially adding to taking away from a likelihood that the spatial pixel images a cancerous cell.

The two analysis methods, spectral analysis and image analysis of a scanned surface, when combined together, potentially provide a highly accurate indication and potentially reduce false positive interpretations.

Size of Probe Influences Use Mode

An aspect of some embodiments is related to a size of a probe, and corresponding medical procedures suited for the size.

In some embodiments, a size of a window such as the scanning window 108 shown in FIG. 1A is on the order of, by way of a non-limiting example, 12 millimeters by 12 millimeters, or more generally, has dimensions of about 0.5 centimeters to about 2.5 centimeters. In some embodiments, such a probe size is optionally used on tissue open to be reached with a probe having such a window. In some embodiments, such a probe size is optionally used in keyhole surgery or laparoscopic surgery, in cases where an opening in tissue is large enough to accept the probe head insertion into an open cut.

In some embodiments, the probe may have a size especially suitable for laparoscopic examination. By way of some non-limiting examples, the probe may have a radius in a range of 2-15 millimeters and a length in a range of 10-30 or even 50 millimeters, or a cross section in a range of 2-15 millimeters by 2-15 millimeter and a length in a range of 10-30 or even 50 millimeters.

In some embodiments, such a probe may be, by way of a non-limiting example, a handheld probe such as the probe 106 shown in FIG. 1A.

In some embodiments, a size of a window such as the scanning window 108 shown in FIG. 1A is on the order of, by way of a non-limiting example, 3-5 millimeters by 3-5 millimeters, or more generally, has dimensions of about 2-3 millimeters to about 7-8 millimeters. In some embodiments, such a probe size is optionally used in laparoscopic, or keyhole surgery.

In some embodiments, such a probe may be, by way of a non-limiting example, a probe capsule, such as will be described below, which is designed to enter into keyhole surgery openings.

In some embodiments, the probe capsule is configured to connect to handles typically used for laparoscopic surgery tools.

In some embodiments, optical components are included in the probe or probe capsule, and data from a spectrometer or hyper-spectrometer built into the probe is communicated to an external processor.

Display

An aspect of some embodiments is related to display.

In some embodiments, indicator light on the probe display indications, including one or more of: scan-starting, scan-ended, cancerous-tissue-detected, indication that an area being viewed includes an overlap with a portion of a previously scanned or viewed area and/or indication that an area being viewed does not include an overlap with a portion of a previously scanned or viewed area, and additional indications as described further below.

In some embodiments, the probe optionally marks a scanned area on a patient's tissue, either in addition to using the indicator lights or independently of using indicator lights.

In some embodiments, the probe optionally marks an area where cancerous tissue has been detected, or is suspected, either in addition to using the indicator lights or independently of using indicator lights.

In some embodiments, an external image display is optionally used, in combination with and/or independently of using the stamping or the indicator light.

In some embodiments, the external image display optionally displays an image of a scanned area, and/or a false-color image of the scanned area.

In some embodiments, the external display displays an aggregate image of scanned areas, optionally stitching together scans from different location. In some embodiments, the external display optionally includes an indication of which areas have been scanned, so that a physician can see if there are voids, areas which should have been scanned and were not scanned.

Contact Non-Contact

An aspect of some embodiments is related to the probe making contact or not making contact with tissue.

In some embodiments, the probe is touched to a patient's tissue.

In some embodiments, a window in the probe flattens the tissue, and optics within the probe are designed to be in focus at a plane of the window touching the tissue.

In some embodiments, a portion of the probe touches and flattens the tissue, and optics within the probe are designed to be in focus at a plane of the flattened tissue. In some embodiments, the portions of the probe designed to touch and flatten the tissue include one or more stamps to marks the tissue.

In some embodiments, the probe is not touched to a patient's tissue, and the optics of the probe are optionally automatically focused onto the tissue. In some embodiments, the automatic focusing uses a range finder. In some embodiments, the automatic focusing uses image analysis to adjust focus.

Medical Procedures

An aspect of some embodiments is related to searching whether cancerous cells or tissue remain in a patient's body after an operation to remove cancerous tissue.

An aspect of some embodiments is related to performing a check to detect whether cancerous cells or tissue remain or have grown in a patient's body a specific period after an operation to remove cancerous tissue, for example a day after, a week after, several weeks, months or years after.

In some embodiments, the spectroscopic analysis replaces and/or is used to augment other types of tests for presence of cancerous tissue.

In some embodiments, the spectroscopic analysis may be performed by a probe entering a natural body opening, without surgical opening of skin. In some embodiments, the spectroscopic analysis may be performed by a probe entering a surgical opening—a small surgical opening followed by spectroscopic analysis may be a desirable price to pay for analysis, even in comparison with other tests for cancerous tissue, or in addition to the other tests.

Some non-limiting examples of medical procedures which potentially benefit from the spectroscopic analysis by a probe and analysis as described herein include:

monitoring and/or elimination of chemotherapy based on clean results by methods as described herein;

inspection for uterine or cervical cancer and/or inspection following an operation for removing uterine or cervical cancer;

inspection for bladder cancer and/or inspection following an operation for removing bladder cancer;

inspection for colon cancer and/or inspection following an operation for removing colon cancer; and

inspection for cancer in the esophagus and/or inspection following an operation for removing cancer from the esophagus.

In some embodiments, the tissue being analyzed is part of a patient's body. In some embodiments, the tissue is an operation cavity, or an operation area, that is, tissue on the patient's body at a location where an operation has removed cancerous tissue.

A potential benefit of the tissue being analyzed being part of a patient's body rather than a sample of excised tissue is that detection of cancerous tissue implies that that the tissue should be excised, at the location where it has been detected—there is no need to analyze, guess or reconstruct where on a patient's body the detected cancerous cells used to be.

In some embodiments, the operation cavity may be small. Some operations nowadays try to keep operation cavities to a minimal size, and in some embodiments, a probe for detecting leftover cancerous tissue is optionally sized to enter into small openings left by the operation.

In some embodiments, the leftover cancerous tissue may include a small amount of cancerous cells, even down to one cell. In some embodiments, a pixel size corresponds to an area of, by way of a non-limiting example, 20 microns by 20 microns. Such a size is on the order of a size one cell, or even smaller, potentially enabling detection down to a level of a single cell.

In some embodiments, detection and/or marking of location of a small amount of cancerous tissue potentially enables removing exactly that tissue. In some cases of removing cancerous cells it is important not to remove too many healthy cells, so as not to damage a remaining organ. Some non-limiting examples of such cases include cancer of the liver and brain cancer.

Some Example Technological Considerations

In some embodiments, an image pixel in a sensor used to detect the cancerous tissue optionally images a small area, so that a spectral and/or hyperspectral signature of the cancerous tissue can potentially possess a signal-to-clutter ratio which enables detection of small amounts of cancerous tissue.

In some embodiments, a field-of-view (FOV) scanned by a system as described herein, by way of a non-limiting example the window 104 shown in FIG. 1A, is approximately on the order of an area which a surgeon would excise if the surgeon is told that there is a remnant of cancerous tissue.

In some embodiments, a number of image pixels and/or hyperspectral pixels is optionally determined by a ratio of the area of the FOV of the system to the area of an image pixel, to provide a beneficial signal-to-clutter ratio and also an area for excising which is similar to the area a surgeon will excise.

In some embodiments, the area scanned by the system is optionally based on a size of an opening into an operational cavity.

In some embodiments, a window for scanning, such as the window 108 shown in FIG. 1A, is optionally sized for entry through an operation opening and/or into an operation cavity.

In some embodiments, a window for scanning, such as the window 108 shown in FIG. 1A, is optionally sized for entry through a natural body opening, without requiring surgical cutting.

In some embodiments, a probe head, such as a head for the probe 106 shown in FIG. 1A, is optionally sized for entry through an operation opening and/or into an operation cavity.

In some embodiments, the above-mentioned window is optionally shaped and/or sized to be places against tissue, so that the tissue is at a location which produces a focused image on an image sensor and/or a hyperspectral sensor.

In some embodiments, the probe head and/or the probe window are detachable from the probe body, so that different sized heads and/or the probe windows may be fitted to a probe body.

In some embodiments, the probe head and/or the probe window are disposable, so that portions of a system as described herein may optionally be used, touching just one patient's tissue before being discarded.

In some embodiments, the probe head and/or the probe window are covered by a transparent disposable cover or film, so that the cover or film may optionally be discarded after touching a patient's.

Further discussion of considerations for which portion of a system may optionally be disposable is provided below.

Guiding Operation of a Probe and/or of Tissue Excision

An aspect of some embodiments is related to guiding operation of a probe, so that the probe scans a target area such as, by way of a non-limiting example, an operation cavity, without missing parts of the area.

An aspect of some embodiments is related to guiding a physician to perform tissue excision where the cancerous tissue has been detected.

In some embodiments, a probe optionally stamps or produces a mark on tissue which has been scanned, so that a physician can see what tissue has been scanned, and perform a next scan adjacent to or even somewhat overlapping with an area which has been scanned.

In some embodiments, the mark optionally marks an outline of the scanned area.

In some embodiments, the mark optionally marks an area in which cancerous tissue has been detected differently from marking an area in which cancerous tissue has not been detected.

In some embodiments, the marking is optionally made when an operator optionally manually initiates the marking.

In some embodiments, the marking is optionally made automatically when the system has completed analyzing a scanned area of tissue.

In some embodiments, a scanning aid is optionally used to guide scanning an operation cavity.

By way of a non-limiting example, the scanning aid is optionally a rigid or semi-rigid guide, such as a ruler, placed on the operation cavity, such that an operator can move a probe by measurable increments.

In some embodiments, the scanning aid is optionally attached to the operation cavity. In some embodiments, the scanning aid attached to operation cavity edges or lips similar to attachment of a medical retractor.

In some embodiments, a physician marks an area or an outline of an area of tissue 320 to be scanned.

In some embodiments, an image analysis unit, whether within a probe or in an external computer, stitches together images of scanned portions of tissue. The image analysis optionally receives an indication of a successful end of a scan and/or analysis of a scanned portion. Of the analysis has not been successful for any reason, the image analysis unit optionally performs one or more of: noting a location of the failed portion, producing an indication of the failure. Some non-limiting examples of a failed scan include: scanned portion not in focus; results of analysis not consistent with expected results (results not showing cancerous tissue and also not benign tissue); a scanned portion which does not include an overlap to any other scanned portion, and so cannot be stitched together to determine whether an entire desired area has been scanned, step-by-step.

In some embodiments, an image analysis unit, whether within a probe or in an external computer, stitches together images of scanned portions of tissue and optionally displays and/or highlights a void, a portion of tissue that the physician missed when stepping a probe and scanning tis sue.

In some embodiments, a processor optionally determines when all the area marked by the physician has been scanned. In some embodiments, an indicator is optionally used to indicate when all the area marked by the physician has been scanned, potentially verifying that all the area marked by the physician has been scanned.

Indicating Detection of Residual Cancerous Tissue

An aspect of some embodiments is related to indicating detection of detection of residual cancerous tissue.

In various embodiments, various ways of indicating detection of cancerous tissue are optionally included. Various such methods are listed below. It is intended that any number of the methods, one or more, may be combined in one embodiment.

One method of indicating detection of cancerous tissue includes operation of an indication light.

One method of indicating detection of cancerous tissue includes stamping a scanned area with a stamp that indicates that cancerous tissue has been detected.

One method of indicating detection of cancerous tissue includes marking an area of cancerous cells on a display which shows an image of tissue that has been scanned.

One method of indicating detection of cancerous tissue includes operation of an indication sound.

One method of indicating detection of cancerous tissue includes displaying an indication icon.

One method of indicating detection of cancerous tissue includes displaying an indication text.

Probe Design

An aspect of some embodiments is related to probe design, including one or more of shape, size and optical design.

In various embodiments, various design considerations are optionally combined. Various such design considerations are listed below. After description of the considerations and a study of the example embodiments described herein, a person of ordinary skill in the art should be able to combine design features in an embodiment.

An example design consideration is whether a probe scans forward, along its longitudinal axis, or sideways to its longitudinal axis. A forward-looking probe is typically suited to scan a bottom of a deep operation cavity and/or a laparoscopic operation cavity. A side-looking probe is typically suited to scan an operation cavity on a patient's body and/or sides of a deep operation cavity and/or a laparoscopic operation cavity. As described here, a portion of a probe may be interchangeable and/or disposable, and a forward-looking probe head may be interchanged with a side-looking probe head and vice versa.

An example design consideration is a size of a probe head and/or scanning window. In such cased where the probe head is intended to enter within an operation cavity, a size of the probe head should be suitable for entering. Such a consideration is appropriate for cases of laparoscopic surgery.

An example design consideration is where along an optical path a separation between a detachable and/or disposable probe head and a probe body should be.

In some embodiments, the separation may optionally be such that a window, such as the window 108 shown in FIG. 1A, and/or a front surface of the probe 106 body are the detachable probe head.

In some embodiments, the separation may optionally be at a slit along the optical path.

In some embodiments, the separation may optionally be along the optical path at a location where light rays are travelling parallel to each other.

An example design consideration is what type of mirror, or what type of scanning mirror, is used. In some embodiment a mechanical scanning mirror may be used. In some embodiments, a MEMS (micro-electro-mechanical systems) mirror may be used. In some embodiments, a MEMS mirror takes up less space in the probe, potentially enabling to produce a smaller probe or probe head.

An example design consideration is an area of a pixel in a scanned area. When an area of a pixel is smaller, a signal of a smaller area of cancerous tissue is optionally detected about background clutter of possible healthy tissue.

An example design consideration is a size of a FOV. When using small pixels, to detect small amounts of cancerous tissue, a FOV may be small. In some embodiments, an array with a large number of pixels is optionally used for scanning, so that the FOV include a reasonably sized area, so as not to slow the scanning procedure unduly.

An example design consideration is a type of spectrometer used for obtaining a spectral signature of the reflected light.

In some embodiments, a spectrometer is used at wavelengths selected for detecting cancerous tissue.

In some embodiments, a hyperspectral imaging sensor is optionally used as a spectrometer. A potential benefit of using a hyperspectral imaging sensor is that such a sensor provides images of a scanned area in a large number of wavelengths, for example 100 different wavelengths. Such a hyperspectral imaging sensor potentially provides several benefits—being commercially available, at a reasonable price, and including wavelength of interest for detecting cancerous tissue. Using a sensor providing a large number of spectral windows or bins potentially enables a more accurate detection of cancerous from non-cancerous tissue signatures, as the signatures each contain more information.

Example Methods of Use

An aspect of some embodiments is related to method of use of a probe for detection of cancerous tissue.

In some embodiments, the probe is optionally used for scanning tissue, or an operation cavity in tissue, and indicating when cancerous tissue is detected.

In some embodiments, the probe is optionally used for marking a location of locations where cancerous tissue is detected in tissue, or in an operation cavity in tissue. In some embodiments, the marking is performed manually, for example by an operator operating a stamp of marking when a probe indicates that cancerous tissue is detected in tissue. In some embodiments, the marking is performed automatically when a probe detects cancerous tissue in tissue In some embodiments, the probe is optionally used for marking an area of tissue which a probe has scanned, potentially enabling an operator to know what has been scanned, and to place a probe in a next position to scan a next area of tissue, based on a marking or stamping of the scanned area.

Data Structure for Scanned Spectral Data

An aspect of some embodiments is related to a data structure used to organize results of a spectral scan of an area of tissue.

In some embodiments, the data structure is a three-dimension matrix, sometimes termed a data cube, having two dimensions corresponding to physical location, such as X and Y, of an element in the data structure, and a third dimension corresponding to a wavelength λ.

In some embodiments, data stored in a data element of the data structure corresponds to a spectral value associated with a pixel at location X, Y at wavelength λ.

In some embodiments, the spectral value may be reflectance, or normalized reflectance, at the wavelength λ, at the location X, Y.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

Before explaining at least one embodiment of the disclosure in detail, it is to be understood that the disclosure is not necessarily limited in its application to the details set forth in the following description or exemplified by the Examples. The disclosure is capable of other embodiments or of being practiced or carried out in various ways.

Reference is now additionally made to FIG. 1B, which is a simplified schematic illustration of a probe and a hosting cradle, according to according to an example embodiment.

FIG. 1B schematically illustrates a probe 10 and a hosting cradle 12, according to certain exemplary embodiments. Probe 10 optionally implements Diffused Reflectance Spectroscopy (DRS) technology in which spectral reflection property or properties contrast between benign and cancerous tissues, potentially providing detection of residual cancerous tissue, and potentially enabling a surgeon to determine a location of the residual cancerous tissues on the body of the patient within the operation cavity.

In some embodiments, the detection is optionally performed during a surgical proceeding. In certain exemplary cases, the probe 10 can be a probe that measures surface without contact.

The probe's pixel size potentially enables the probe 10 to access small features, as small as a single cell.

In some embodiments, the probe 10 is designed to scan an area approximately sideways relative to a longitudinal axis of the probe 10.

In some embodiments, the probe 10 is designed to scan an area approximately on-axis relative to a longitudinal axis of the probe 10. An on-axis scanning probe potentially enables the probe 10 to measure recessed features within an operation cavity that may not be accessible otherwise.

In some embodiments, the probe 10 optionally includes a casing 14.3 that facilitates holding the probe 10 in a hand of a user.

In some embodiments, the probe 10 optionally includes a disposable scanning head 14.1 configured to scan a designated area for the residual cancerous tissue. The disposable scanning head 14.1 can have different forms, widths and geometries to facilitate providing a surgeon with the flexibility to select a scanning head that will best fit the geometry of the operation cavity and area being scanned on the patient body. One or several scanning heads 14.1 can be used during a medical procedure such as an operation, with each scanning head 14.1 optionally being disposed after completion of the scan on the patient body, optionally avoiding the exposure of the scanning heads 14.1 to sterilization procedures that other surgery tools are exposed to.

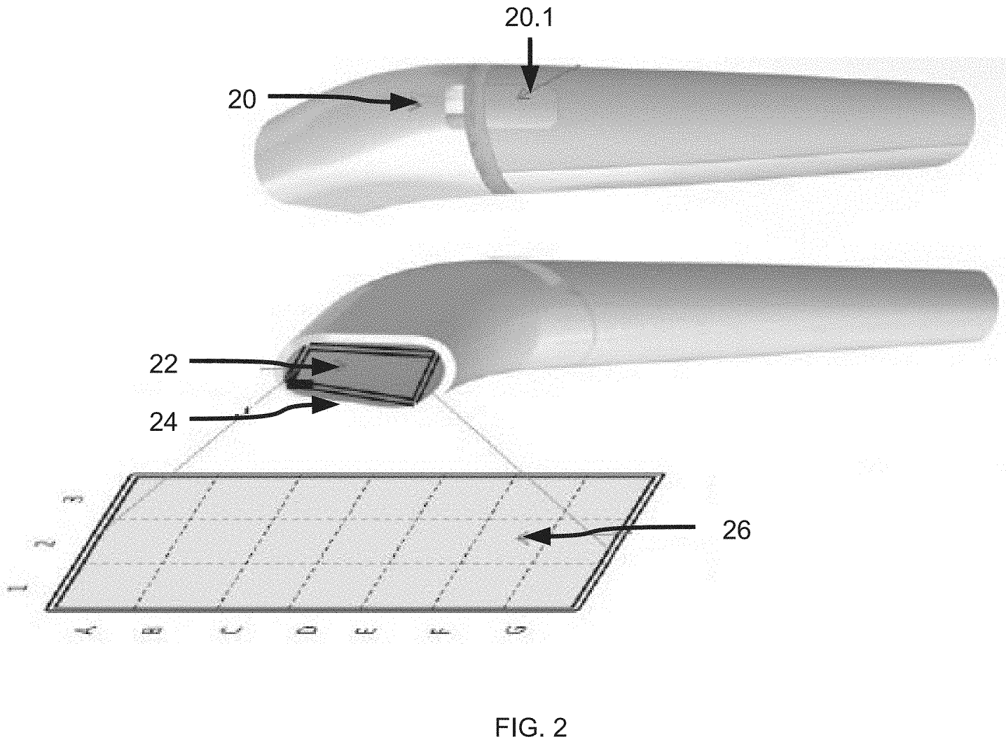

FIG. 1B schematically illustrates two exemplary scanning head configurations that show a forward scanning head 14.2 and a side scanning head 14.1. The disposable scanning head optionally includes an optical system having an assortment of optical elements such as one or more of a mirror, a slit, a beam-splitter, and/or the like, a scanning window (see reference 22 in FIG. 2) and stamping elements (see reference 24 in FIG. 2) located on an external perimeter of the scanning window.

In some embodiments, a reusable handle or casing 14.3 containing electronic hardware, which may optionally include a processor executing software, a rechargeable power battery, optical system components and/or the like.

Reference is now additionally made to FIG. 1C, which is a simplified schematic illustration of a probe and an external display, according to according to an example embodiment.

FIG. 1C is intended to show options for an external display which, In some embodiments, may optionally be used to display information.

FIG. 1C shows a probe 120 with a scanning window 124; an optional base 122; an optional display 126 attached to the base 122; and an optional display 128.

The optional display 126 attached to the base 122 may be a cell phone or a tablet or a dedicated display. The optional display 128 shown not attached to the base 122 may be a computer display, or a tablet, or a dedicated display.

The optional display(s) are optionally in communication with the probe 120, either by wired communication as is known in the art, or by wireless communication as is known in the art.

Reference is now additionally made to FIG. 2, which is a simplified schematic illustration of elements and indicators of a probe according to some example embodiments.

FIG. 2 shows that in some embodiments, a reusable handle 14.3 as shown in FIG. 1B can optionally also include one or more indication lights 20. Function(s) of the indication light(s) 20 will be described below.

FIG. 2 also shows that in some embodiments, a cradle 12 may be provided, in which the probe 10 is optionally positioned while not in operation. In some embodiments, the probe 10 and the cradle 12 optionally include contacts 15A 15B for charging the probe 10.

In some embodiments, the cradle 12 optional design is made to have minimal impact on any crowded operation room. In some embodiments, the probe 10 has a size, light indicators and weight of a handheld device, which is easy to use, intuitive and agile.

In some embodiments, during a medical procedure such as an operation, a surgeon removes a tumor mass based on current procedures, methods and hospital rules. Usually such procedures, also known as Standard Operating Procedures (SOP) which call for examining the resected tumor for clean margins. At this stage the operation cavity from where the tumor was resected, is exposed. The surgeon optionally uses the probe 10 to identify whether any residual cancerous tissues remains within the operation cavity on the patient's body.

The surgeon, after turning on the probe, optionally places a scanning window 22 within the operational cavity, optionally on the cavity surface, and initiate a scan, optionally by using an Initiate Scan Button 20.1.

In some embodiments, an optional yellow indicator light 20 optionally lights up while scanning, to indicate that a scan is being performed by probe.

In some embodiments, after a scan is completed, the yellow light turns off.

In some embodiments, the surgeon does not move the probe 10 until processing is over and a green or red indicator light 20 turns on and off again. In such embodiments the green or red indicator lights 20 optionally indicate no cancerous tissue was detected, or the cancerous tissue was detected, respectively.

In some embodiments, the surgeon moves the probe to its next position within the operational cavity only once the green or red lights turn off.

In some embodiments, turning on of the red light 20, indicating detection of cancerous tissue, is optionally followed by stamping elements 24 marking boundaries of the cancerous tissues, as described in below, detected within a Field Of View (FOV) of the scanning window 22.

In some embodiments, one or more indicator lights 20 on the probe 10 may optionally include:
Blue—probe 10 in operation, the probe will turn on (blue light on) as it is removed from its cradle (12).
Yellow—scan in progress.
Green—No cancerous tissues were detected within the scanning window during the present scan.
Red—Cancerous tissues were detected within the scanning window area during the present scan.

In some embodiments, following red light and stamping, the surgeon may optionally remove detected cancerous tissues located within the stamped marks on the operation cavity surface left by the stamping elements.

In some embodiments, removing the tissues is optionally followed by repeated scan of the cavity area until the green light is the only indication, meaning the cancerous tissues were completely removed from the surface of the operation cavity, and/or that no cancerous tissues were detected found within the scanned area.

An example of guiding a surgeon to a location of detected cancerous tissue is now described.

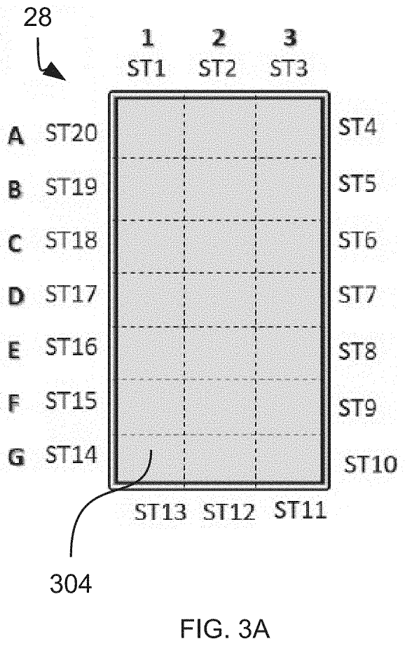

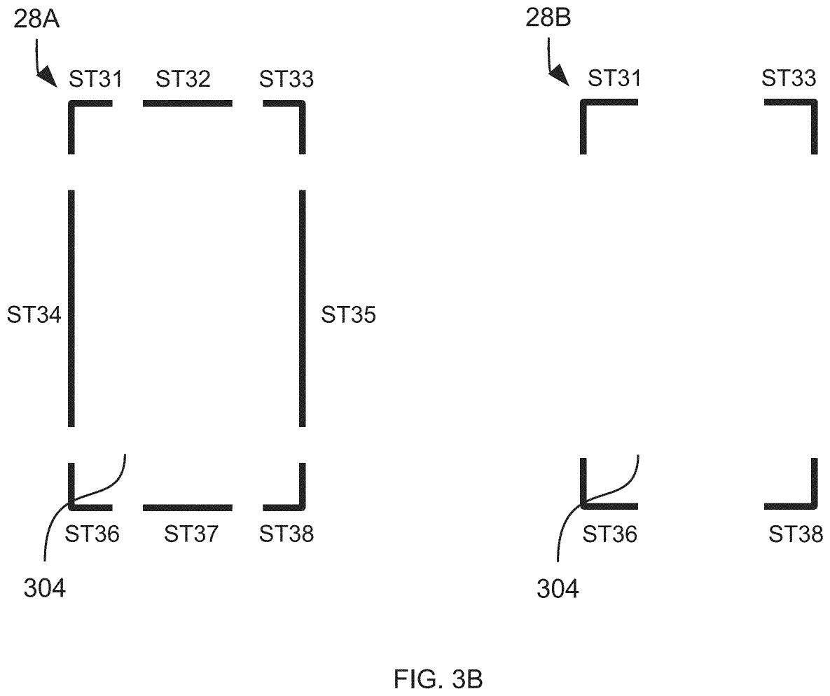

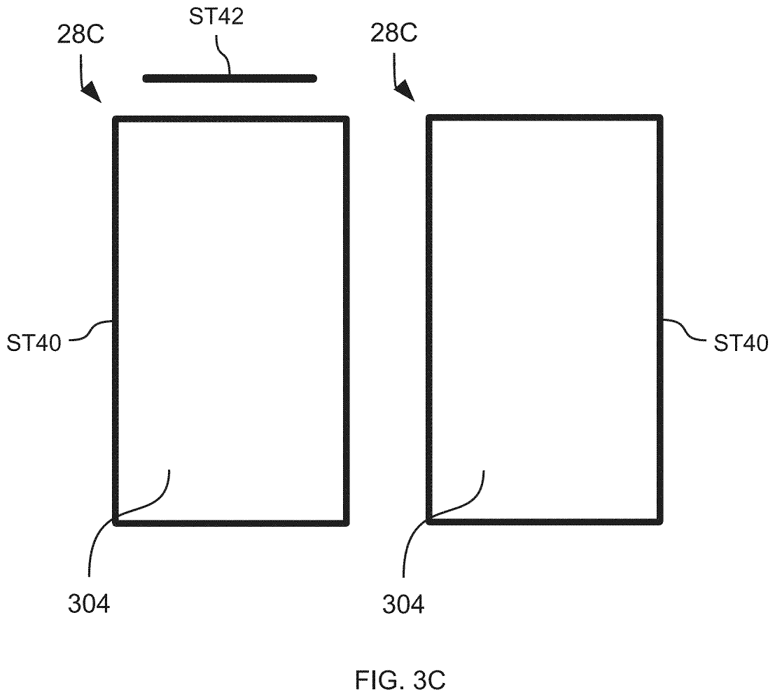

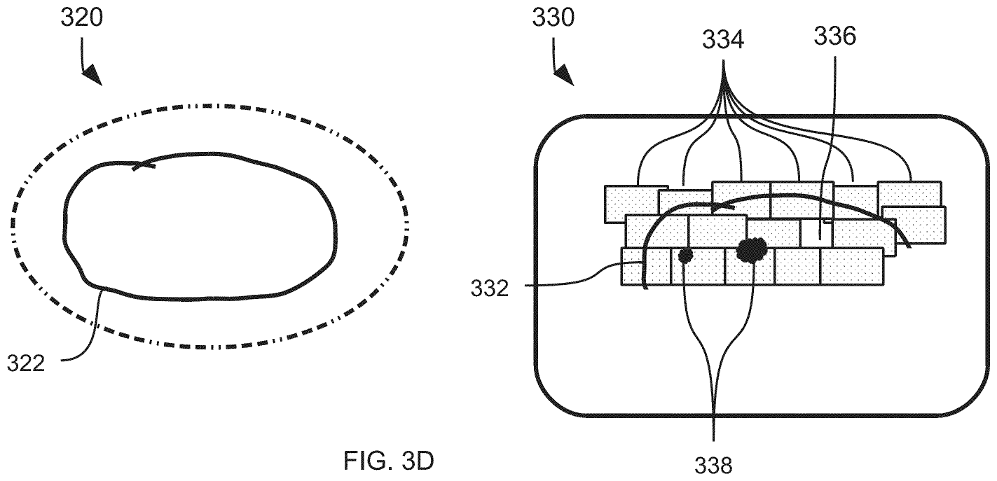

In some embodiments, a scanned area under the scanning window is divided into 21 sectors (reference 26 in FIG. 2 and reference 28 in FIG. 3A).

In some embodiments, each sector location is optionally identified by a pair of matrix elements A-G along one axis of the matrix, and matrix elements 1-3 along a perpendicular axis of the matrix, for example: sector A1, sector E3 etc.

Reference is now additionally made to FIG. 3A, which is a simplified schematic illustration of a scanning window with surrounding stamping elements, according to some example embodiments.

FIG. 3A shows a mapping 28 of stamping elements for marking portions of a scanned area.

In some embodiments, a scanning window 304 is optionally surrounded by 20 stamping elements ST1-ST20.

It is noted that the example of FIG. 3A should not be considered limiting. Additional arrangements of stamps are conceived, which may divide an area scanned into dimensions other than 3 columns by 7 rows, for example 2 columns by 2 rows, stepping either the number of columns, or the number of rows, or both, up to 10 columns or rows or even more.

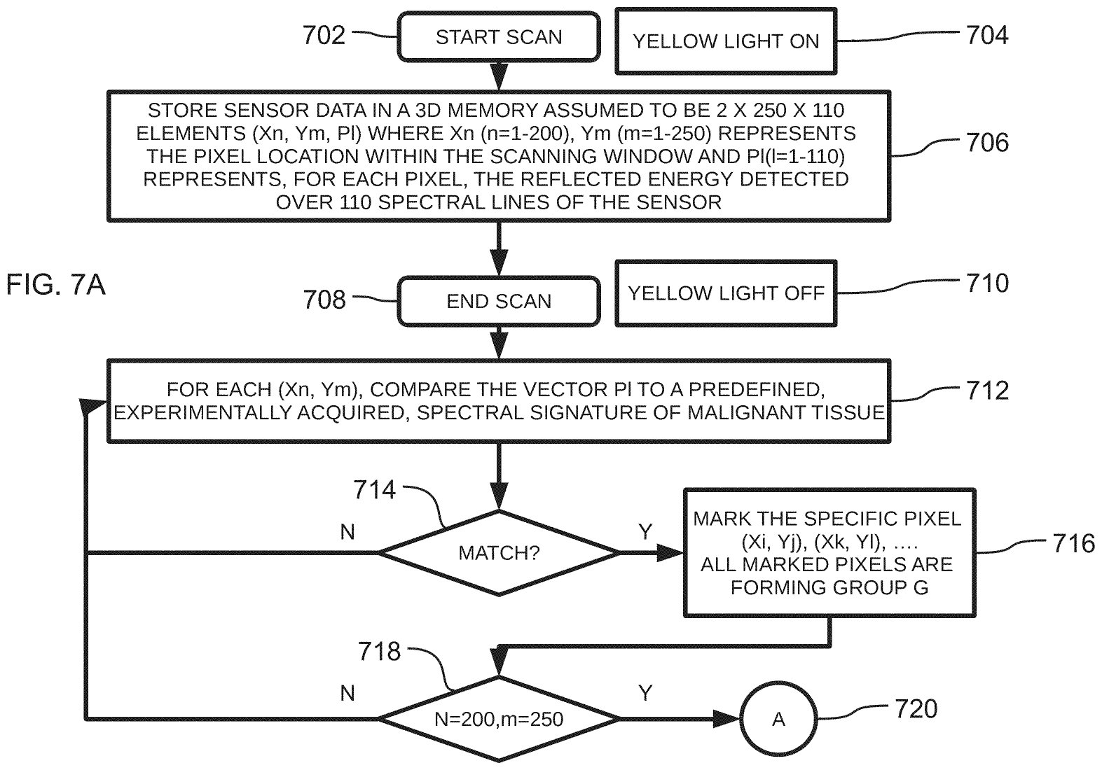

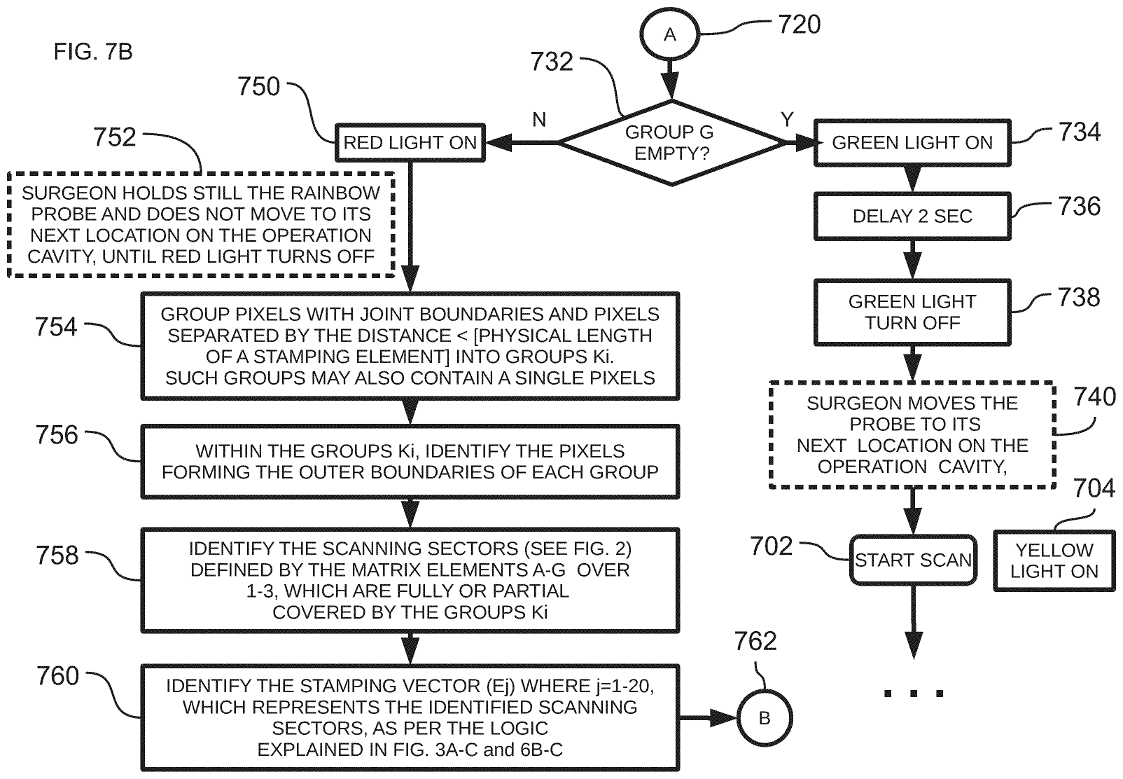

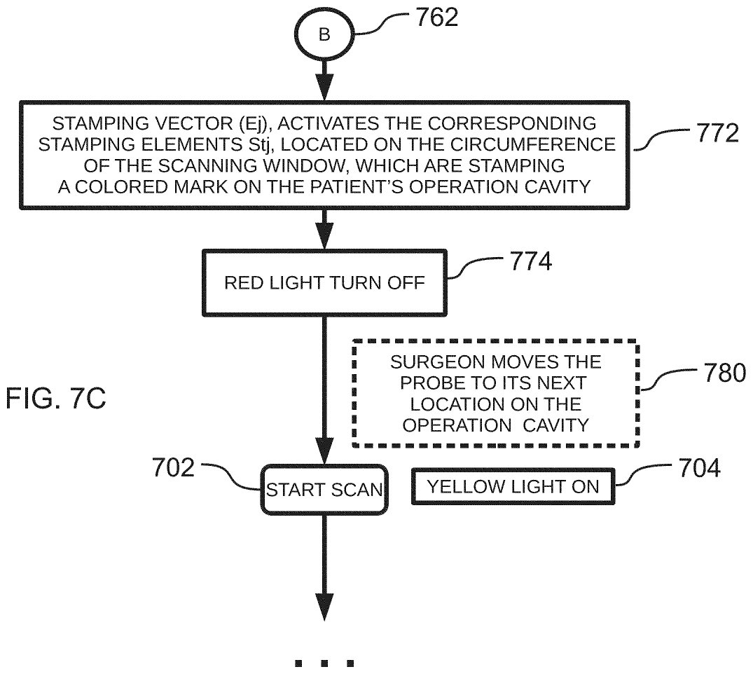

In some embodiments, the stamping elements are optionally activated by an algorithm such as described in FIG. 7, which identifies potential presence and location of residual cancerous tis sue.

In some embodiments, the stamps corresponding to the cancerous tissue location leaves a colored mark within the operation cavity, guiding the surgeon for further resection.

Some non-limiting examples:
1) Cancerous tissue identified in sector C2, the activated stamping elements are: ST2, ST12, ST6, ST18.
2) Cancerous tissues identified in sectors E2, E3, the activating stamping elements are ST16, ST8, ST2, ST3, ST12, ST11.
3) Cancerous tissue identified in sectors A1, B1, the activated stamping elements are ST1, ST19, ST20.

Reference is now additionally made to FIG. 3B, which is a simplified schematic illustration of a scanning window with surrounding stamping elements, according to some example embodiments.

FIG. 3B shows a first stamp for optionally marking a scanned area.

In some embodiments, a scanning window 304 is optionally surrounded by 8 stamping elements ST31-ST38. By way of a non-limiting example, four of the stamping elements ST31 ST33 ST36 ST38 mark corners of the scanning window 304, and four of the scanning elements ST32 ST34 ST 35 ST37 mark portions of sides of the scanning window 304.

In some cases, when cancerous tissue is detected within the scanning window 304, all the 8 stamps ST31-ST38 are optionally operated, marking a rectangle surrounding an area where cancerous tissue has been detected. Since a surgeon typically removes tissue all around a location where cancerous tissue has been detected, in such embodiments the entire scanning window 304 is surrounded by a stamp.

In some cases, when cancerous tissue is not detected within the scanning window 304, only some of the 8 stamps ST31-ST38 are optionally operated, for example only the four corner stamps ST31 ST33 ST36 ST38, marking corners of a rectangle surrounding the area where cancerous tissue has not been detected. Such marking optionally marks the area which has been scanned, and potentially indicates to an operator where to place the probe for scanning an adjacent area, or a slightly overlapping neighboring area.

Reference is now additionally made to FIG. 3C, which is a simplified schematic illustration of a scanning window with surrounding stamping elements, according to some example embodiments.

FIG. 3C shows a first stamp for optionally marking a scanned area.

In some embodiments, a scanning window 304 is optionally surrounded by a stamping element ST40. By way of a non-limiting example, the stamping element ST40 marks the scanning window 304.

In some cases, when cancerous tissue is detected within the scanning window 304, a marking stamp ST42 is optionally operated, marking a line or bar next to the area where the cancerous tissue has been detected. Since a surgeon typically removes tissue all around a location where cancerous tissue has been detected, in such embodiments the entire scanning window 304 is surrounded by a stamp.

In some cases, when cancerous tissue is not detected within the scanning window 304, the stamping element ST42 is not operated, only the stamping element ST40 marks a rectangle surrounding the area where cancerous tissue has not been detected. Such marking optionally marks the area which has been scanned, and potentially indicates to an operator where to place the probe for scanning an adjacent area, or a slightly overlapping neighboring area.

Reference is now additionally made to FIG. 3D, which is a simplified schematic illustration of a display showing areas which have been scanned, according to some example embodiments.

FIG. 3D show an example method by which a physician may be shown which areas of tissue have been canned and which areas may have been missed, or still require scanning.

In some embodiments, a physician marks an area or an outline of an area 322 of tissue 320 to be scanned. The marking may be performed with a medically safe color which is visible to a probe sensor, as is known in the art.

When the physician scans a portion of the tissue, a sensor images the portion and optionally displays the image 334 of the portion on a display 330. When the physician scans an adjacent portion of the tissue, the sensor images the adjacent portion, and optionally display the adjacent image 334 on a display 330.

In some embodiments, the outline of the area 322 is visible in the displayed images, optionally also as an outline 332, and potentially assists a physician to track what portions of the tissue have been scanned.

In some embodiments, an image analysis unit, whether within the probe or in an external computer, stitches together the image portions, optionally based on overlapping portions of the adjacent images. In some embodiments, the image analysis unit optionally uses the image of the outline of the area 322 to stitch together the image portions.

A potential advantage of displaying the scanned tissue may be that a physician can see a void 336, a portion of tissue that the physician missed when stepping a probe and scanning tissue.

In some embodiments, a processor optionally determines when all the area 322 marked by the physician has been scanned. In some embodiments, an indicator is optionally used to indicate when all the area 322 marked by the physician has been scanned, potentially verifying that all the area 322 marked by the physician has been scanned.

In some embodiments, the display receives indication of where cancerous cells were detected, and optionally display location 338 of the cancerous cells on the display 330. Displaying the location 338 cancerous cells may on the display 330 may be performed independently of additional modes of indicating detection of cancerous cells, such as stamping, described elsewhere herein, and using an indicator light, described elsewhere herein. A physician may choose to perform a scan to detect cancerous cells keeping eyes on the tissue only (stamps may mark cancerous locations), eyes on the tissue and the probe (indicator light may mark cancerous locations), or occasionally look at the display and see cancerous locations and/or area covered by the scanning process.

### Example Principles of Operation:

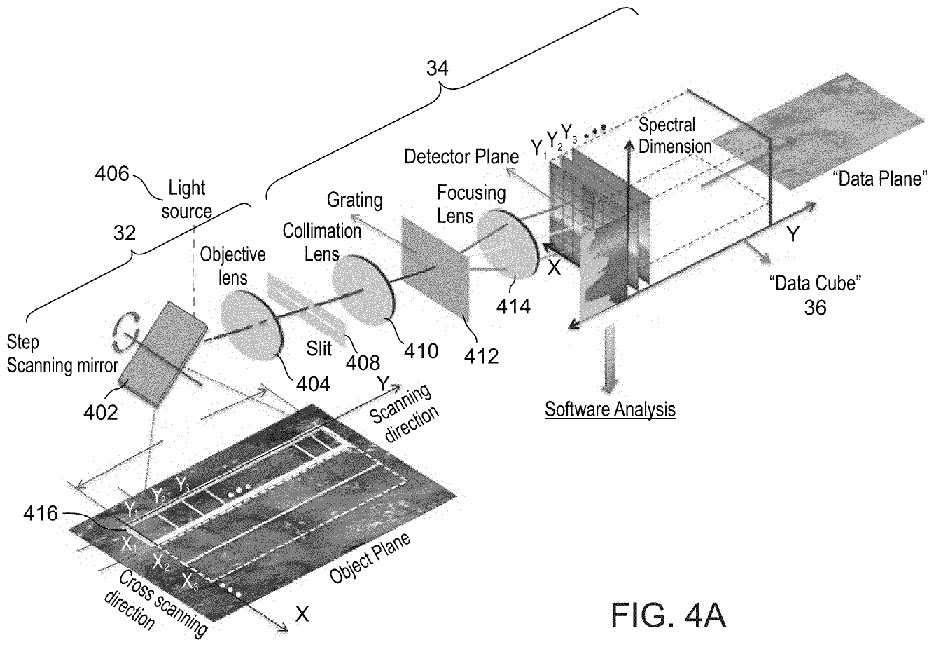

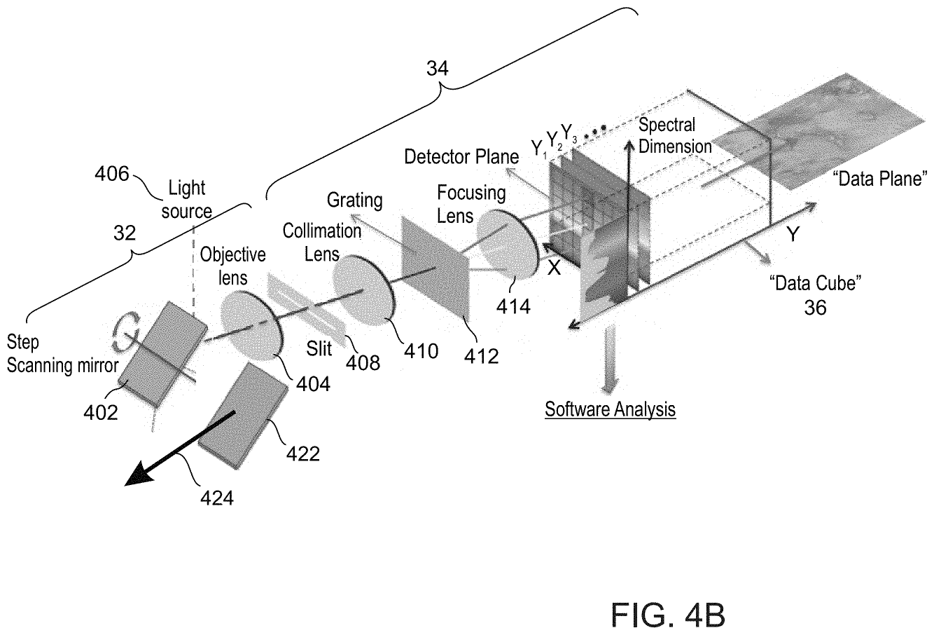

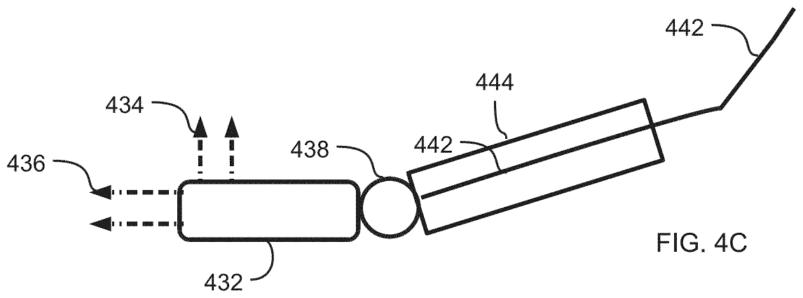

### a) An Example Optical System—FIG. 4A

Reference is now made to FIG. 4A, which is a simplified schematic illustration of a method of operating a probe according to an example embodiment.

FIG. 4A schematically illustrates light flow in an optical system which includes a scanning head (SH) 32 and a spectral measurement system (SMS) (34).

### 1) An Example Scanning Head (SH) 32:

In some embodiments, the scanning head 32 may optionally be a disposable element.

In some embodiments, the scanning head may optionally contain one or more of the following sub-elements: a scanning mirror 402 (optionally a step scanning mirror 402); an objective lens 404; a light source 406 and a slit 408.

In some embodiments, additional sub-elements of the optical system, such as a collimation lens 410, a grating 412 and a focusing lens 414, are included in an optical system back-end. In some embodiments, the optical back-end is part of a probe handle.

In various embodiments the SH 32 may have different forms, potentially to be selected by a surgeon based on geometry of an operational cavity to be scanned.

In a description of FIG. 1B an option of a side scanning SH is described. However, another possibility can optionally be a forward scanning SH.

In some embodiments, in order to keep similarity with the side scanning SH, a possible implementation of a forward scanning SH can optionally have an additional, optionally fixed, mirror (not shown), mounted opposite the scanning mirror 402, where a plane of the additional mirror is at 45 degrees to the optical axis of the optical system elements. The image coming in from a forward scanning window, reflected on the additional mirror, is now be scanned by the scanning mirror 402, in the same way and with same optical elements as in the side scanning SH.

An example scanning principle—At each step of the scanning mirror 402 (translated by an angle of movement of the scanning mirror surface), a single pixel size in a width of Yn (for example approximately 0.1 mm) of the scanned tissue, is reflected on the slit 408. A cross-scan resolution X is potentially determined by a spatial separation of spectral sensor elements (not shown in FIG. 4A), as later described.

With each step of the scanning mirror 402, a light source 406 within the SH illuminates scanned tissue 416, and reflected light is reflected back to the scanning mirror 402 and then to the objective lens 404, which focuses the light on the slit 408, behind which is the spectral measurement system (SMS) 34.

Reference is now made to FIG. 4B, which is a simplified schematic illustration of a method of operating a probe according to an example embodiment.

FIG. 4B is intended to show one example optical design of a forward scanning probe, where FIG. 4A showed an example optical design of a side scanning probe.

FIG. 4B shows the references shown in FIG. 4A, to which a mirror 422 has been added. The scanning mirror 402 scans an area which is redirected by the mirror 422 to be forward, along a direction of the optical axis between the scanning mirror 402 and the slit 404.

It is noted that the scanned tissue 416 of FIG. 4A, not shown in FIG. 4B, would be shown in FIG. 4B forward, along a direction of the optical axis between the scanning mirror 402 and the slit 404.

Reference is now made to FIG. 4C, which is a simplified schematic illustration of a probe according to an example embodiment.

FIG. 4C is intended to show a probe 432 sized and configured to connect to a laparoscopic tool handle 444.

FIG. 4C shows the probe 432 connected to a tool handle 444 by an optional hinge 438.

In some embodiments, the probe 432 includes optic components up to and including a spectral sensor, and data from the sensor is optionally communicated, either by wire 442 as shown in FIG. 4C, or wirelessly, to an external processing unit for performing data analysis and/or display as described elsewhere herein.

The probe 432 may be a forward scanning 436 probe, or a side scanning 434 probe, as described elsewhere herein.

In some embodiments, the probe 432 may have a size especially suitable for laparoscopic examination. By way of some non-limiting examples, the probe 432 may have a radius in a range of 2-15 millimeters and a length in a range of 10-30 or even 50 millimeters, or a cross section in a range of 2-15 millimeters by 2-15 millimeter and a length in a range of 10-30 or even 50 millimeters.

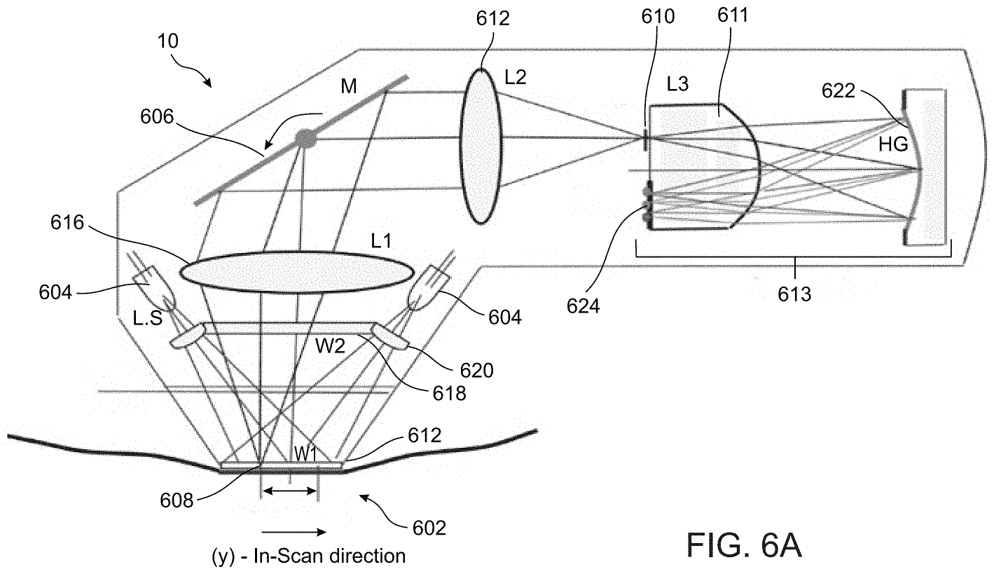

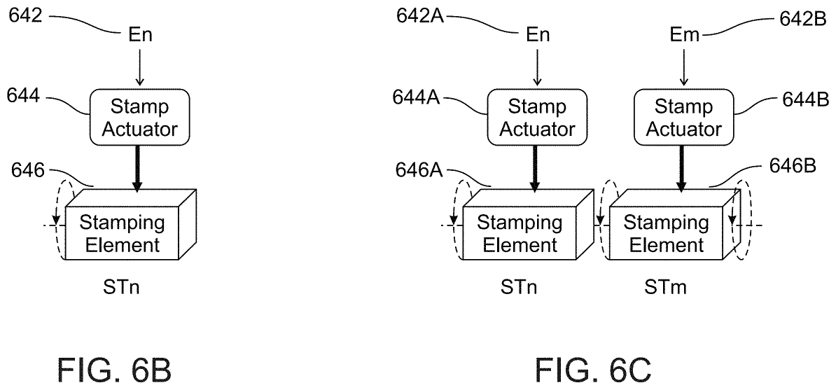

### 2) An Example Implementation of a Probe, Including an Example Scanning Head (SH) and an Example Spectral Measurement System (SMS) FIG. 6A:

Reference is now made to FIG. 6A, which is a simplified illustration of an optical design of a probe according to an example embodiment.

FIG. 6A illustrates one optional implementation among many, for an optical design of a probe 10. The probe 10 is not limited to this implementation only, which is explained hereinafter for the sake of implementation clarity.

FIG. 6A shows an area of tissue 602 for scanning, a first window W1 616, one or more light source(s) 604, an optional second window W2 618, optional lens(es) 620 for the light source(s) 604, an optional collimating lens L1 616, a scanning mirror 606, an optional converging lens L2 612, a slit 610, an optional third lens L3 611, a grating HG 622, and a sensor array 624.

In some embodiments, the scanning mirror 606 is a step scanning mirror.

In some embodiments, the grating 622 is a holographic grating.

In some embodiments, the one or more light source(s) 604 are optionally wide spectral light source(s).

The scanned area of tissue 602 is illuminated by the one or more light sources 604. For a given angle of the scanning mirror M 606, a width Δy 608 of the tissue 602 is imaged at the entrance slit 610 of the spectrometer 613. A magnification m produced by the lenses L1 616 and L2 612 is m=f(L2)/f(L1), where f(L2) and f(L1) are the focal-length of the converging lens 612 and the collimating lens 614, respectively.

Y is a coordinate over the tissue, set along the in-scan direction.

In some embodiments, a Dyson spectrometer 613 is used, as shown in FIG. 6A. The Dyson spectrometer has a robust and a compact structure with the L3 lens 611 and a reflecting holographic grating (HG) 622.

The spectrometer 613 forms a spectrally dispersive image of the slit 610 over a multiline-output-sensor 624. Each line of the sensor 624 receives an image of the tissue—Δy 608 at a different wavelength, so that all the lines of the sensor simultaneously record an entire spectral structure.

The process of spectral imaging is optionally performed step by step by the scanning mirror 606, until the entire scanning-area 602 of the tissue is fully scanned.

In some embodiments, the spectrum analyzer may optionally include a prism-grating-prism design, as described in a publication titled “Hyperspectral prism-grating-prism imaging spectrograph”, written by Mauri Aikio of VTT (Technical Research Centre of Finland) in a dissertation for the degree of Doctor of Technology at the University of Oulu.

### 3) An Example Spectral Measurement System (SMS)

As mentioned earlier, the probe 10 design is not limited to the design of the system described herein which is one option, among many SMS designs of the probe 10. A purpose of a system as described herein is to measure energy content within a specified range of wavelengths, composing the reflected light beam.

The reflected light passing through a slit is focused (as parallel lines) on a grating (Diffraction Grating) by a collimating lens. The grating diffracts the incoming light (which is the light reflected from the scanned tissue) into its spectral components. Each of the wavelengths, composing the incoming light, is diffracted to a different angle. A focusing lens, such as the lens L3 611, located at a distance of its focal length from the grating, transfers diffracted beams of different wavelengths, into parallel beams illuminating corresponding spots on the sensor plane, as explained hereafter.

A sensor unit, such as the sensor 624, is configured to measure light. In some embodiments, the sensor is configured to sense light within a range of 420 to 990 nanometer.

In some embodiments, a sensor plane includes a series of CMOS receptors, measuring light energy over, for example, 110 wavelength ranges within a span of 420-990 nanometers, with a 5 nanometer resolution or less.

In some embodiments, the number of CMOS receptors in the sensor plane can be on the order of 200 or more, with a spatial separation of less than 1 micrometer.

Data measured by the sensor plane includes dimensions represented as [Pλ, Xm], where Pλ is the energy within a wavelength range or wavelength bin, λ and Xm is a Pixel m, as presented by reference 34 in FIG. 4A.

Pλ is optionally measured per each pixel, thereby defining a spectral signature of a spatial pixel within a scanned area.

In some embodiments, for each step of the scanning mirror 402, a line of a width Yn of at least 200 pixels (in the cross-scan direction), including spatial pixels of a width Xm, in an operational cavity is scanned.

The reflected light energy from this line is diffracted on the bi-dimensional sensor plane (Pλ, X).

In some embodiments, using the above example method, spectral content (also named spectral signature) of the reflected light from each spatial pixel on the scanned surface, is measured and optionally stored in a 3D memory. The data is also named a Data Cube 36.

### b) Data Analysis and Processing Algorithms

Medical research articles have indicated that light reflected from cancerous tissue, its energy content within each particular wavelength across the measured spectrum of wavelengths (spectral signature) is distinguishably different from the spectral signature of light reflected from benign tissue. Such a principle is used in analyzing the spectral signature of the reflected light from single pixels, of the scanned tissue, over which the scanning window is placed.

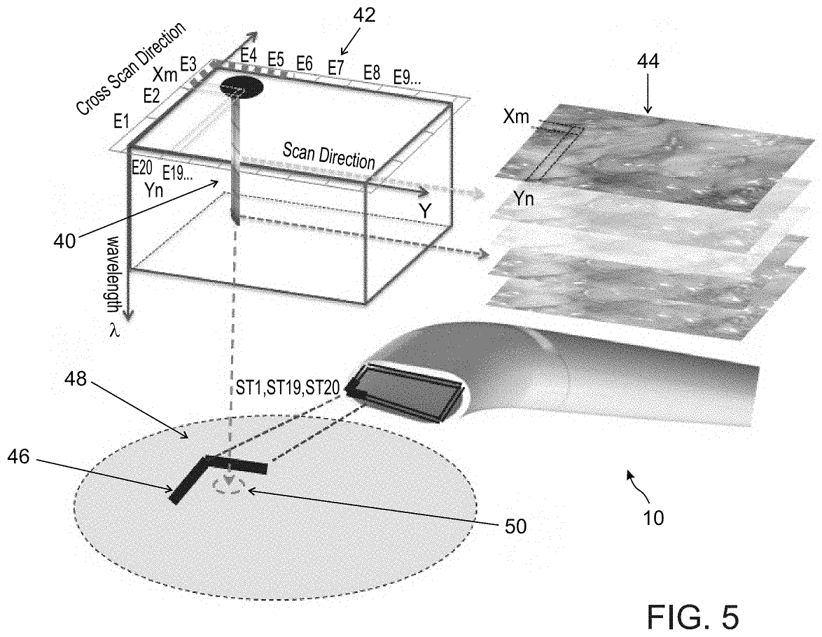

Reference is now made to FIG. 5, which is a simplified illustration of a probe scanning tissue and data produced by the probe, according to an example embodiment.

FIG. 5 shows a probe 10 scanning an operation cavity 48, and a data cube 40 which includes two-dimensional images 44 at different wavelengths. The two-dimensional images 44 are shown as a stack of two-dimensional images 44, each two-dimensional image at a different wavelength bin.

The Data Cube (reference 36 in FIG. 4A and reference 40 in FIG. 5) includes the following elements: for each pixel whose location within the scanning window is defined by (Xm, Yn), where Y is the scanning axis and X is the cross-scanning axis, an associated 3rd axis is λ, which is the wavelength.

In some embodiments, the wavelengths span from 420 nanometer to 990 nanometer. The energy reflected from each spatial pixel, is spread over sensor plane spectral lines, forming the spatial pixel's spectral energy content.

In some embodiments, the spectral energy content of the spatial pixels may optionally be analyzed as will be described in relation to FIG. 7.1.

The spectral energy content of the spatial pixels is optionally analyzed for a possible match with spectral signature(s) related to the existence of cancerous tissue within a particular spatial pixel.

In some embodiments, a location of at least one spatial pixel identified with cancerous tissue content is optionally marked as such. In some embodiments, a red indicator light on the Probe is optionally turned on.

In some embodiments, a location of at least one spatial pixel identified with cancerous tissue content is optionally automatically marked as such. In some embodiments, a red indicator light on the Probe is optionally automatically turned on.

Reference is now made to FIGS. 6B and 6C, which are simplified illustrations of methods for marking cancerous tissue according to some example embodiments.

A pixel or cluster of pixels identified as including cancerous tissue optionally generates one or more activation signal(s) En (En, Em . . . ), which activate stamping actuation signals, which cause one or more stamping element(s) (STn, STm, . . . ) to mark location(s) of cancerous tissue in a scanned area.

FIG. 6B shows one activation signal En 642, which activates a stamping actuation signal 644, which cause a stamping element STn to mark a location of cancerous tissue in a scanned area, and/or to mark which area has been scanned.

FIG. 6C shows activation signals En 642A, Em 642B, . . . , which activate stamping actuation signals 644A 644B . . . , which cause stamping elements STn 646A, STm 646B, . . . to mark a location or locations of cancerous tissue in a scanned area.

Reference is now made to FIGS. 7A-7C, which show a method of processing sensor data according to some example embodiments.

Example software algorithms are shown in FIGS. 7A, 7B and 7C.

FIGS. 7A, 7B and 7C show a method which includes:

FIG. 7A shows:

starting a scan (702), which optionally includes turning a yellow—scanning—indication light on (704);

storing sensor data in a 3D memory (706), assumed to be 2×250×110 elements (Xn, Ym, Pl) where Xn (n=1-200), Ym (m=1-250) represents the pixel location within the scanning window and Pl (1=1-110) represents, for each pixel, the reflected energy detected over 110 spectral lines of the sensor;

ending the scan (708), which optionally includes turning the yellow scanning indication light off (710);

for each (Xn, Ym), comparing the vector Pl to a predefined, optionally experimentally acquired, spectral signature of malignant tissue (712);

determining whether the vector matches (716) the spectral signature;

if NO, returning to above action marked (712);

if YES, marking the specific pixel (Xi, Yj), (Xk, Yl), . . . . all marked pixels are forming group G (716);

repeating actions 702-716 by incrementing N for all Ym and incrementing M for all Xn, determining whether n=200 and m=250 (718);

if NO, returning to above action marked (712);

if YES, going to “A” in FIG. 7.2 (720).

FIG. 7B shows:

At “A”, determining whether group G is empty (732);

if YES:
turning green light ON (734);
delaying (736), optionally for 2 seconds;
turning green light OFF (738);
optionally, surgeon moves the probe to its next location on the operation cavity (740); and
returning to above actions marked 702 and optionally 704 in FIG. 7A;

if NO:
turning red light ON (750);
optionally, surgeon holding still the probe and does not move to its next location on the operation cavity, until red light turns off (752);
grouping pixels with joint boundaries and pixels separated by the distance <[physical length of a stamping element] into groups Ki (754). Such groups may also contain a single pixels;
within the groups Ki, identifying the pixels forming the outer boundaries of each group (756);
identifying the scanning sectors (see FIG. 2) defined by the matrix elements A-G over 1-3, which are fully or partial covered by the groups Ki (758);
identifying the stamping vector (Ej) where j=1-20, which represents the identified scanning sectors, as per the logic explained in FIGS. 3A-C and 6B-C (760);
going to “B” in FIG. 7.3 (762).
FIG. 7C shows:
At “B” (762) performing the following:
stamping vector (Ej) (772), activating the corresponding stamping elements Stj, located on the circumference of the scanning window, which are stamping a colored mark on the patient's operational cavity;
turning red light OFF (774);
optionally, surgeon moving the probe to its next location on the operation cavity (780);
and
returning to above actions marked 702 and optionally 704 in FIG. 7A.

A pixel or cluster of pixels which are determined to include cancerous tissue, optionally generate activation signals (En, Em . . . ), which optionally activate the stamping elements (STn, STm, . . . ) accordingly, as described with reference to FIGS. 3A-C and 6B-C.

In some embodiments, the stamping is optionally used as a method of guiding the surgeon to the location of the detected cancerous tissues.

Reference is again made to FIG. 5.

In some embodiments, activated stamping elements mounted on outer perimeters of the scanning window, optionally stamp a colored mark 46 on an operational cavity 48.

In some embodiments, following the stamping a red light on the probe 10 is optionally turned off, and the surgeon may remove the probe 10, moving the probe to its next location. The surgeon optionally places the probe 10 at a new location in the operational cavity. In some embodiments, the new location may overlap, or partially overlap, with a previous location.

In some embodiments, the stamp designates to the surgeon, a perimeter of residual cancerous tissue 50.

In some embodiments, the surgeon will remove the marked tissue.

In some embodiments, after removing designated cancerous tissues, the surgeon repeats the scanning of the operation cavity, and repeats removing residual cancerous tissues, until no red light is turned on in a scan over the operational cavity.

In some embodiments, after removing designated cancerous tissues, the surgeon repeats the scanning of the operation cavity, and repeats removing residual cancerous tissues, until only the green light turns on marking that no more residual cancerous tissues were detected within the scanned operational cavity.

The determination of tumor's clean margins during surgical resection is a challenging task. A complete removal of malignant tissue while maximizing conservation of healthy tissue has been a critical factor, impacting not only local recurrence and survival of a patient, but also the patients' post-operative functionality and need for repeated surgeries.

In some cases, among women who underwent lumpectomy (breast conserving surgery or partial mastectomy), 30%-60% had to go through repeated surgery, since their doctors received information about breast tissue that was removed, only after the initial surgery was completed and the patient was in recovery process or even sent home. Such repeated surgeries are placing patients' well-being at risk, compromise the patients' quality of life and become a heavy burden on health services providers, insurance companies and hospitals. A paramount decision a surgeon makes during surgery concerns margin's resection.

Margins are evaluated today mostly through in-vitro pathological resected tumor tissue assessment, after surgery or during surgery, by a pathological laboratory assessing a removed tumor mass by a method of Frozen Slicing or other method, which are time consuming methods, during which a patient may be under continuous anesthesia and the operation room may be idle. This method poses an additional major problem: correlating a location of the malignant cells identified on resected tissue with their exact location on the patient's body. Such a problem potentially intensifies in cases where a tumor is removed by shaves or slices and not as one-block. Variation in surgeons' skills and experience may also playing a role in successful identification and complete removal of a malignant tumor during a first surgery session.

The probe is potentially a tool for the surgeon, designed in some embodiments as a handheld probe, which can potentially provide objective, real-time, in-vivo, intraoperative detection and designation of tumor margins and malignant residuals. The probe potentially increases a success rate of complete tumor removal and clean margins at a first surgery.

Hyper-spectral imaging, originating from remote sensing, has been initially explored by NASA, and followed by aerospace industries, for various applications including vegetation and water resource control, food quality etc. mainly for earth observation applications.

For medical applications, hyper-spectral imaging offers non-invasive tissue diagnosis. Light delivered to biological tissue undergoes multiple scattering from inhomogeneity of biological structures and absorption primarily in hemoglobin, melanin and water, as it propagates through the tis sue.

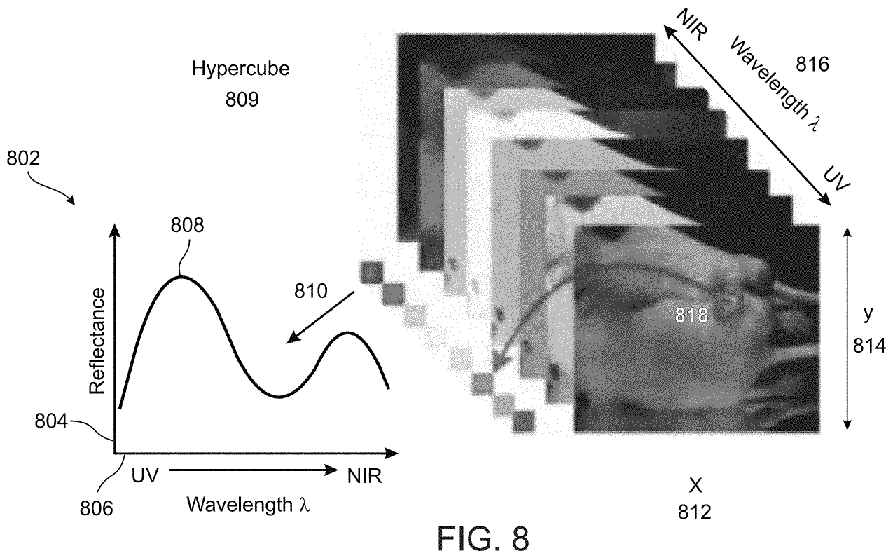

Reference is now made to FIG. 8, which is a simplified illustration of a spectral graph and different wavelength spatial images which were used to produce the graph according to an example embodiment.

FIG. 8 shows a graph 802 showing a spectral signature 808 utilized for detection of cancerous tissue, according to certain exemplary embodiments.

The graph 802 has an X-axis 806 of wavelength λ and a Y-axis 804 of reflectance intensity.

FIG. 8 also shows a hypercube 809, or data cube 809, which includes two-dimensional images of a scanned tissue, having an X-axis 812 and a Y-axis 814, the two-dimensional images at different wavelengths 816.

FIG. 8 shows a process of a location 818 on a two-dimensional image corresponding 810 to a location on the spectral signature 808.

Tissue is illuminated and reflected light is collected from each pixel within a spatial surface. Each pixel is designated a coordinate value, for example, as a (X, Y) value. The reflected light energy per each wavelength of reflected light within the spectrum provides a graph, which shows the “spectral signature” of the specific pixel, from which the reflected light is collected. Cancerous tissue potentially differs from benign tissue by one or more parameters which result in a different spectral signature. For example, an amount of blood vessels, carrying a larger amount of oxyhemoglobin, and a higher tissue density in cancerous tissue relative to benign/normal tissue. Oxyhemoglobin, as a lead differentiator, has a unique spectral signature resulting from the oxygen absorption line. In addition, densities of the tissue results in different light returns form the tissue, due to different scattering processes.

There are also numerous papers showing the usage of spectral signature in the cancer research, for example:
i. Review of methods for intraoperative margin detection for breast conserving surgery—Journal of Biomedical Optics 23(10), 100901 (October 2018)
ii. Diagnosis of breast cancer using elastic-scattering spectroscopy: preliminary clinical results—Journal of Biomedical Optics 5(2), 221-228 (April 2000)
iii. Detecting positive surgical margins: utilization of light-reflectance spectroscopy on ex vivo prostate specimens—BJU Int 2016; 118: 885-889

The surgeon scans an operation cavity surface after removing the tumor mass. The probe 10 uses diffused spectral imaging technique which creates a three-dimensional data cube. Two dimensions are aligned according to a X, Y coordinate system imposed onto a spatial image of a scanned area in high resolution, for example in a 30 micrometer by 30 micrometer area, and a third axis representing a spectral wavelength. The spectral signature of an image pixel is obtained, for example, by placing a separate spectrometer to correspond to pixels of the scanned area.

In some embodiments, an example embodiment includes an imaging system in which, per each single scan, the scanned surface can be of a size of 12 millimeter by 12 millimeter, providing a result of a combined analysis, through implementation of algorithms, analyzing spectral signature, optionally per each pixel in a high resolution image, obtained during scanning.

In some embodiments, a scan is optionally performed on a body of a patient within an operation cavity.

In some embodiments, image analysis of the scanned area is optionally performed. In some embodiments, the image analysis is optionally performed at a pixel resolution, for example for pixels each having a field-of-view of 30 micrometer by 30 micrometer area. In some embodiments, structure of the spatial images is optionally analyzed by an Artificial Intelligence and/or Machine Learning algorithms, to search for morphological structures indicating benign or cancerous tissue. In some embodiments, known structural images of benign and cancerous tissues are used as the primary data base for such analysis.

In some embodiments, the two unrelated analysis methods, spectral analysis at the pixel level and image analysis of the scanned surface, when combined together at a pixel level, may potentially provide a better indication and may reduce false positive interpretations.

In some embodiments, false positive interpretations may result in excessive healthy tissue removal. Such interpretation in case of breast cancer surgery may then result in more complex breast reconstruction. However, in cases of liver or brain cancer, excessive healthy tissue removal could potentially be more critical to the patient's well-being.

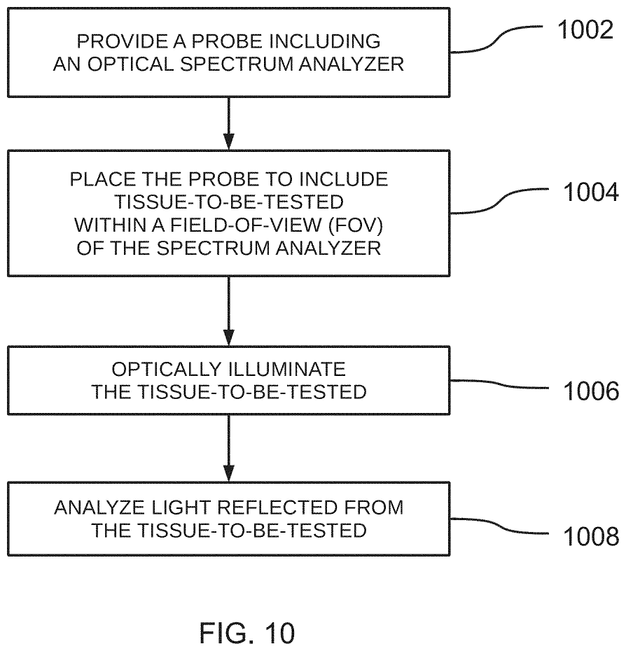

Reference is now made to FIG. 10, which is a simplified flow chart illustration of a method for detecting cancerous tissue according to an example embodiment.

The method of FIG. 10 includes:

providing a probe including an optical spectrum analyzer (1002);

placing the probe to include tissue-to-be-tested within a Field-Of-View (FOV) of the spectrum analyzer (1004);

optically illuminating the tissue-to-be-tested (1006); and

analyzing light reflected from the tissue-to-be-tested (1008),

thereby determining whether or not cancerous tissue is detected in the FOV.

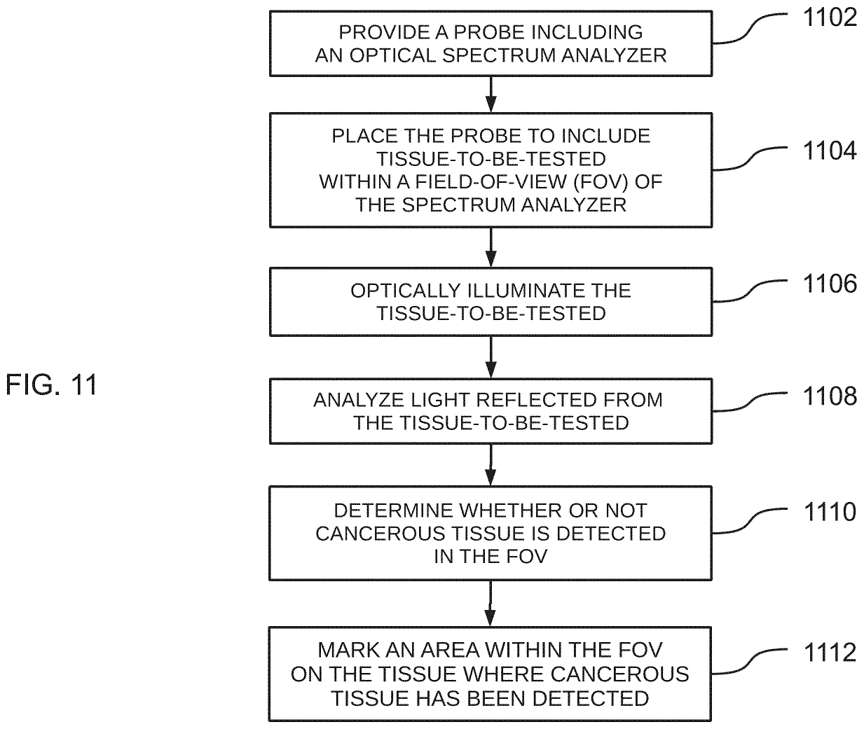

Reference is now made to FIG. 11, which is a simplified flow chart illustration of a method for marking cancerous tissue according to an example embodiment.

The method of FIG. 11 includes:

providing a probe including an optical spectrum analyzer (1102);

placing the probe to include tissue-to-be-tested within a Field-Of-View (FOV) of the spectrum analyzer (1104);

optically illuminating the tissue-to-be-tested (1106);

analyzing light reflected from the tissue-to-be-tested (1108),

determining whether or not cancerous tissue is detected in the FOV (1110); and

marking an area within the FOV on the tissue where cancerous tissue has been detected (1112).

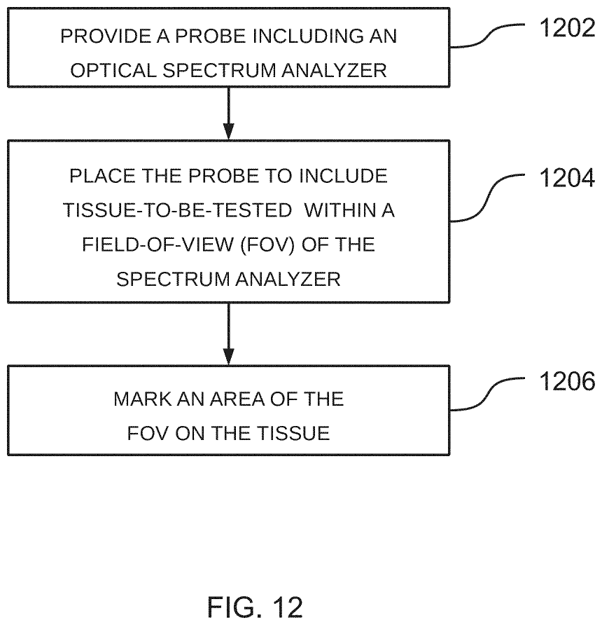

Reference is now made to FIG. 12, which is a simplified flow chart illustration of a method for guiding scan of tissue according to an example embodiment.

The method of FIG. 12 includes:

providing a probe including an optical spectrum analyzer (1202);

placing the probe to include tissue-to-be-tested within a Field-Of-View (FOV) of the spectrum analyzer (1204);

marking an area of the FOV on the tissue (1206).

It is expected that during the life of a patent maturing from this application many relevant spectrometers will be developed and the scope of the term spectrometer is intended to include all such new technologies a priori.

It is expected that during the life of a patent maturing from this application many relevant hyper-spectrometers will be developed and the scope of the term hyper-spectrometer is intended to include all such new technologies a priori.

As used herein with reference to quantity or value, the term “about” means “within ±50% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

### EXAMPLES

Reference is now made to the following examples, which together with the above descriptions illustrate some embodiments of the disclosure in a non-limiting fashion.

As part of a medical research under Helsinki charter, conducted by Prof. Hanoch Kashtan, Director of Chirurgical Department at Rabin Medical Center, a laboratory instrumentation setup was used, functionally imitating a single pixel measurement, by which resected breast cancer tumor examinations are received from an operation room. Using algorithms, a clear differentiation between cancerous and healthy tissues can be achieved. Those results were in correlation with the pathologist designation of the same sampling points.

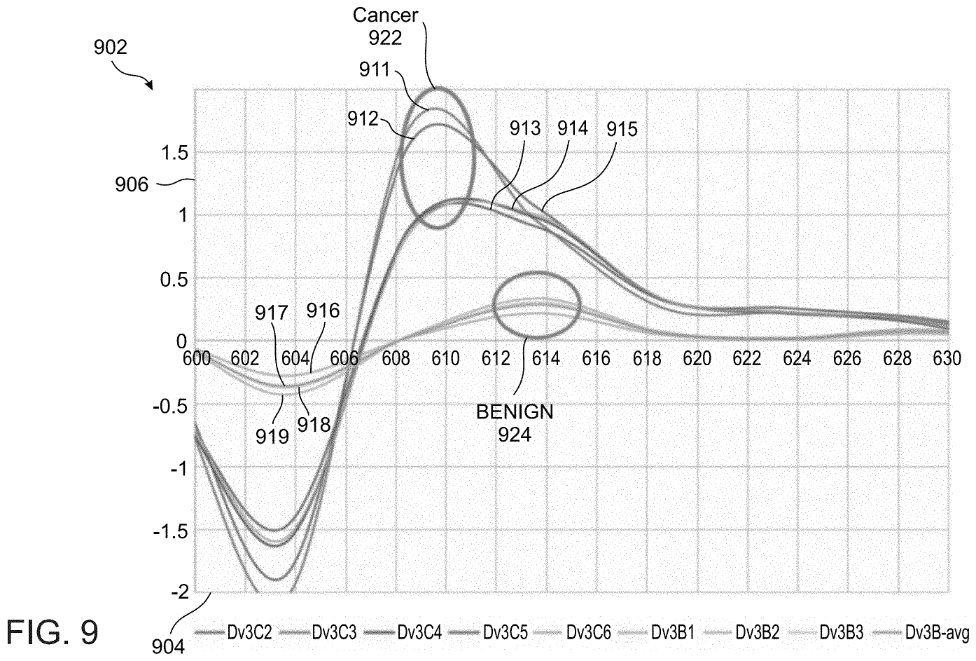

Reference is now made to FIG. 9, which is a graph showing experimental spectra of several locations in tissue scanned according to an example embodiment.

FIG. 9 shows a graph 902. The graph 902 has an X-axis 904 of wavelength λ, a Y-axis 906 of normalized reflectance at wavelength λ. Each line 911 912 913 914 915 916 917 918 919 in the graph corresponds to reflectance values from one pixel in a two-dimensional image of tissue.

The graph 902 includes data scanned from a tissue sample of one patient.

The graph 902 demonstrates 5 lines 911 912 913 914 915, associated with 5 pixels in the scanned image of the tissue, showing a spectral signature 922 associated with cancerous tissue, and 4 lines 916 917 918 919, associated with 4 pixels in the scanned image of the tissue, showing a spectral signature 924 not associated with cancerous tissue.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-43. (canceled)

44. A method for detecting cancerous tissue, the method comprising:
providing a probe comprising a hyperspectral analyzer;
placing the probe to include tissue-to-be-tested within a Field-Of-View (FOV) of the spectrum analyzer;
optically illuminating the tissue-to-be-tested;
capturing a plurality of images of a surface of the tissue-to-be-tested, each image of the plurality of images at a different wavelength window;
analyzing a spectrum of a plurality of pixels at a corresponding location in the plurality of images;
determining a type of tissue at the corresponding location; and
determining whether or not cancerous tissue is detected in the FOV.

45. The method according to claim 44, wherein the determining the type of tissue in the pixel comprises performing image analysis of at least one of the plurality of images based on morphological structures indicating benign or cancerous tissue determined to exist in the at least one image.

46. The method according to claim 44, wherein the determining a type of tissue in the pixel comprises:
allocating a value to the pixel based on the type of tissue;
producing a synthetic image from the values of the pixels; and
performing image analysis of the synthetic image.

47. The method according to claim 45, wherein:
the performing image analysis comprises performing image analysis of a plurality of images of the FOV captured at a plurality of wavelength bands;
the plurality of wavelength bands is a group of wavelength selected from an entire range wavelengths bands reflected from the tissue-to-be-tested;
machine learning is used to select the plurality of wavelength bands.

48. The method according to claim 44, wherein the determining whether or not cancerous tissue is detected in the FOV comprises combining a likelihood of detection of cancerous tissue based on image analysis and a likelihood of detection of cancerous tissue based on diffuse spectroscopy analysis.

49. The method according to claim 44, wherein the analyzing a spectrum of each pixel is performed on an imaged area smaller than 0.1 mm×0.1 mm.

50. The method according to claim 44, wherein placing the probe comprises placing the probe to flatten the tissue-to-be-tested.

51. The method according to claim 44, and further comprising automatically marking an area of the FOV on the tissue.

52. The method according to claim 44, and further comprising marking an area of tissue-to-be-tested using a color visible to the spectrum analyzer.

53. The method according to claim 44, and further comprising displaying images captured by the probe.

54. The method according to claim 53, wherein the displaying comprises displaying the marking.

55. The method according to claim 44, and further comprising indicating whether all of the area of tissue-to-be-tested has been imaged by the probe.

56. The method according to claim 54, wherein the displaying comprises displaying where cancerous tissue has been detected.

57. A system for detecting cancerous tissue, the system comprising:
a hand-held probe comprising:
an illuminator; and
a hyperspectral imager; and
an indicator for indicating whether cancerous tissue has been detected in reflected spectrum analyzed by the optical spectrum analyzer,
wherein the system comprises a hyperspectral analyzer configured to:
capture a plurality of images of a surface of the tissue-to-be-tested, each image of the plurality of images at a different wavelength window;
analyze a spectrum of a plurality of pixels at a corresponding location in the plurality of images;
determine a type of tissue in the pixel; and
determine whether or not cancerous tissue is detected in the FOV.

58. The system according to claim 57, wherein the probe comprises a detachable probe head detachable from a handle of the probe, and wherein the probe head comprises a component for contacting tissue.

59. The system according to claim 57, wherein the probe comprises a disposable probe head detachable from a handle of the probe, and wherein the probe head comprises a component for contacting tissue.

60. The system according to claim 57, wherein the probe comprises a stamp for marking tissue at a border of the window.

61. The system according to claim 60, wherein the probe comprises an actuator for automatically stamping tissue based on whether cancerous tissue has been detected in reflected spectrum analyzed by the optical spectrum analyzer.

62. The system according to claim 58, wherein the probe is configured to detach the detachable probe head at a window for transferring reflected light from illuminated tissue to the spectrum analyzer.

63. The system according to claim 62, wherein the probe is configured to detach the detachable probe head at a slit along the optical path from the window to the spectrum analyzer.
